(19) United States
(12) Patent Application Publication
(10) Pub. No.: US 2009/0138988 A1
(43) Pub. Date: May 28, 2009

(54) MODIFICATION OF PLANT DISEASE RESISTANCE

(76) Inventors: Keri Wang, London (CA); Choong-Min Ryu, Daejeon (KR); Kirankumar Mysore, Admore, OK (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 12/176,962

(22) Filed: Jul. 21, 2008

Related U.S. Application Data
(60) Provisional application No. 60/951,203, filed on Jul. 21, 2007.

(51) Int. Cl. C12N 15/54

(52) U.S. Cl. 800/279

(57) ABSTRACT

The invention provides methods of increasing resistance to disease in a plant. Also provided are plant steroid biosynthesis enzyme coding sequences, including sequences for squalene synthase, constructs comprising these sequences, plants transformed therewith and methods of use thereof. Methods for producing a plant with increased disease resistance are also provided. In certain aspects of the invention, methods for producing plants transformed with the nucleic acids are provided, the transformed plants exhibiting improved disease resistance.

This application claims the priority of U.S. Provisional Application Ser. No. 60/951,203, filed Jul. 21, 2007, the entire disclosure of which is incorporated herein by reference.

### BACKGROUND OF THE INVENTION

1. Field of the Invention

The invention relates generally to the field of molecular biology. More specifically, the invention relates to methods of modulating disease resistance and susceptibility in a plant.

2. Description of the Related Art

Nonhost resistance, shown by a plant species to a specific parasite or pathogen, is the most common and durable form of plant resistance to diseases (Heath, 2000). Although there has been progress in plant science generally, nonhost pathogen resistance remains poorly understood in contrast with host resistance, shown by specific genotypes within an otherwise susceptible host species. Host resistance is often governed by single resistance (R) genes, the products of which directly or indirectly interact with specific elicitors produced by avirulence (avr) genes (Flor, 1971; Hammond-Kosack and Jones, 1997). Considerable progress has been made in the understanding of gene-for-gene resistance (R-avr interactions; Martin, 1999). Some plant genes involved in nonhost disease resistance have also been identified. However, it is still not clear why a pathogen fully virulent to one plant species is nonpathogenic to others.

Squalene is the biochemical precursor to all steroids. Plant sterols, sometimes referred to as phytosterols, are structural components of plant cell membranes. SQS encodes squalene synthase, an enzyme that catalyzes the first committed step of the sterol biosynthetic pathway. Sterol biosynthesis and accumulation is suppressed in response to pathogen or elicitor challenge in various plant species (Threlfall and Whitehead, 1988; Vogeli and Chappell, 1988; Zook and Kuc, 1991; Fulton et al., 1993).

Plant disease outbreaks can cause great crop losses, which can create economic hardship for farmers and famine in areas that are predominantly dependent upon only a few crops for subsistence. Generally, plant disease control is implemented by breeding and using resistant cultivars selected or developed for this purpose. Genetic engineering of crop plants to confer broad resistance to several pathogens, however, remains a promising objective. Thus, a better understanding of nonhost resistance mechanisms, and additional strategies to protect plants against plant disease, are needed.

### SUMMARY OF THE INVENTION

In one aspect, the invention provides a method for increasing disease resistance in a plant, the method comprising expressing in the plant a nucleic acid encoding squalene synthase and/or sterol methyltransferase. Increased resistance to P. syringae pv. glycinea and P. syringae pv. tomato specifically, is provided. Also provided are nucleic acid sequences that when stably incorporated into the plant genome as part of a construct, increase disease resistance in a plant. Sequences of the present invention include SEQ ID NO:1-11; nucleic acid sequences encoding the polypeptides of SEQ ID NO: 12-14; and nucleic acid sequences having at least 95%, 90%, or 85% sequence identity of any nucleic acid sequences listed above. Similarly, sequences of the present invention include nucleic acid sequences complementary to, or that hybridize to SEQ ID NO: 1-11 and/or nucleic acid sequences encoding the polypeptides of SEQ ID NO: 12-14.

The invention also includes methods of making a plant with increased disease resistance as compared to a plant that has not undergone the claimed method, such as a plant lacking a heterologous squalene synthase coding sequence 1. Plants of the invention can be monocots or dicots. In one embodiment, the plant is either Arabidopsis thaliana or Nicotiana benthamiana. Promoters used with the method of the invention may be inducible, organelle-specific, tissue-specific, cell-specific, developmentally-specific, pest and/or pathogen-inducible or constitutive. The invention provides a construct that is introduced into a plant comprising a nucleic acid sequence such as SEQ ID NO: 1-11; nucleic acid sequences encoding the polypeptides of SEQ ID NO: 12-14, and also provides for the construct comprising at least one additional sequence chosen from the group consisting of: a regulatory sequence, a selectable marker, a screenable marker, a secretable marker, a leader sequence, and a terminator. The construct may be inherited from a parent plant of the plant. The plant may also be an R₀ transgenic plant.

In yet another aspect, the invention provides a recombinant vector comprising an isolated polynucleotide of the invention. The nucleic acid sequence may be in sense orientation and may be an antisense oligonucleotide of a coding sequence provided by the invention. Such an antisense oligonucleotide may, but need not necessarily comprise the full length of a coding sequence provided by the invention. In certain embodiments, the recombinant vector may further comprise at least one additional sequence chosen from the group consisting of: a regulatory sequence, a selectable marker, a leader sequence and a terminator. In further embodiments, the additional sequence is a heterologous sequence and the promoter may be constitutive, developmentally-regulated, organelle-specific, inducible, inducible, tissue-specific, constitutive, cell-specific, seed specific, pest and/or pathogen-inducible or germination-specific promoter. The recombinant vector may or may not be an isolated expression cassette.

In still yet another aspect, the invention provides a seed of a transgenic plant of the invention, wherein the seed comprises the selected DNA. The invention also provides a host cell transformed with such a selected DNA. The host cell may express a protein encoded by the selected DNA. The cell may have inherited the selected DNA from a progenitor of the cell and may have been transformed with the selected DNA. The cell may be a plant cell.

In still yet another aspect, the invention provides a method of increasing the pest and/or disease resistance of a plant comprising introducing into the plant a nucleic acid encoding a steroid biosynthesis enzyme such as squalene synthase. In a method of the invention, up-regulating squalene synthase may be carried out by introducing a recombinant vector of the invention into a plant. Such introducing may be carried out by plant breeding using a transgenic plant as starting material or directly by genetic transformation. Down-regulating may also be carried out, including by use of antisense oligonucleotides or by RNA interference technology, as is known in the art.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1. shows P. syringae pv. glycinea induced symptoms on the leaves of silenced N. benthamiana plants. Two to three weeks after inoculation with TRV containing individual gene sequences, the upper leaves of gene silenced N. benthamiana plants were challenged with non-host pathogen P. syringae pv. glycinea (type I) at the concentration of 2×10⁷ cfu/ml. Pictures were taken at two and seven days post exposure to the pathogen.

FIG. 2. Hypersensitive reactions of the silenced N. benthamiana plants with the identified genes to the non-host pathogen P. syringae pv. tomato T1 (type II). Two to three weeks after inoculation with TRV containing individual gene sequences, the upper leaves of silenced N. benthamiana plants were challenged with P. syringae pv. tomato T1 at the concentration of 2×10⁷ cfu/ml. Pictures were at one day and two days after exposure to the pathogen.

FIG. 3 illustrates bacterial accumulation in some of the silenced N. benthamiana plants when challenged with the nonhost pathogen P. syringae pv. tomato T1. Two to three weeks after inoculation with TRV containing individual gene sequences, the upper leaves of silenced N. benthamiana plants were challenged with GFPuv labeled P. syringae pv. tomato T1 (type II nonhost pathogen) at a concentration of 2×10⁵ cfu/ml by vacuum infiltration. Pictures were taken 4 days after inoculation under long wave length UV light. The green spots in leaves indicate bacterial accumulation.

FIG. 4. shows growth of non-host pathogens, P. syringae pv. glycinea (4A) and P. syringae pv. tomato T1 (4B) in the silenced N. benthamiana plants. Two to three weeks after inoculation with TRV containing individual gene sequences, the upper leaves of gene silenced N. benthamiana plants were vacuum infiltrated with P. syringae pv. glycinea or P. syringae pv. tomato T1 at the concentration of 2×10⁴ cfu/ml. Leaf disks were taken from six infiltrated plants at different time intervals, ground, and plated.

FIG. 5. NbSQS silenced N. benthamiana plants are compromised for both host and nonhost resistance. Observation of growth of GFPuv labeled nonhost pathogens (P. syringae pv. tomato T1, P. syringae pv. glycinea and X. campestris pv. campestris) and host pathogen (P. syringae pv. tabaci) in NbSQS silenced plants and control was carried out under long wave length UV light 4 days after infection (upper two panels; FIG. 5A). Bacterial number was examined 3 days after inoculation at 5×10⁴ cfu/ml (lower four panels; FIG. 5B.

FIG. 6. Electrolyte leakage of the silenced N. benthamiana plants without infection (left panel) and wild type plants infected by various pathogens (right panel). Two leaf discs of the NbSQS silenced N. benthamiana plants and control plants were shaken in 5 ml of milliQ water for 3 hrs at room temperature. The ion conductivity of the solution was measured as electrolyte conductivity in the apoplast. The total electrolyte conductivity of the leaf discs was determined after autoclaving. The ratio of apoplast conductivity to total conductivity was used as electrolyte leakage of cell membrane. To measure electrolyte leakage caused by host and nonhost pathogens, wild type plants were vacuum infiltrated with each pathogen at the concentration of 5×10⁴ cfu/ml. Cell leakage was measured every 24 hrs after inoculation.

FIG. 7. Metabolite profiling of apoplast extracted from NbSQS silenced N. benthamiana and control plants. Plant leaves were excited and vacuum infiltrated in milliQ water for 1 min. The excess water on leaf surface was removed gently. Apoplast was extracted from the infiltrated leaves by centrifugation at 500 g for 10 min at room temperature and used for GC-MS analysis.

FIG. 8. Bacterial growth in minimal medium containing 5% of harvested apoplastic fluid. Bacterial cells were collected by centrifugation of overnight culture, washed twice; resuspended in minimal growth medium (MGM) and used to inoculate the fresh MGM with or without apoplast. Bacterial growth at 30° C. was determined by measuring the optical density at 600 nm (OD₆₀₀) of the bacterial culture every hour for 14 hours. Dark blue line: MGM only; purple line: MGM containing 5% of apoplast extracted from control N. benthamiana; yellow line: MGM containing 5% of apoplast extracted from NbSQS silenced N. benthamiana.

FIG. 9. A. Phenotype of Arabidopsis AtSQS1 RNAi lines compared to the vector control. B. Relative expression of AtSQS1 in the transgenic AtSQS1 RNAi lines examined by real-time PCR. C. Symptoms of Arabidopsis AtSQS1 RNAi lines infected with a nonhost pathogen P. syringae pv. tabaci. Photographs were taken four days after inoculation at the concentration of 106 cfu/ml. D. Bacterial growth in the transgenic AtSQS1 RNAi lines. Bacterial numbers were determined by plating serial dilutions of leaf extracts 3 days after inoculation.

FIG. 10. shows relative expression of SQS1 and SQS2 in the transgenic lines of Arabidopsis (10A) and N. benthamiana (10B). SQS mRNA expression in Arabidopsis was determined by semi-quantitative RT-PCR, while SQS mRNA expression in N. benthamiana was examined by real-time quantitative RT-PCR. The transgenic lines are indicated at the top or bottom of the graph. Y axis (10B) shows relative gene expression levels of SQS to the vector control (value 1). Error bars were derived from 3 technical replicates.

FIG. 11. Brief summary of the pathway of sterol biosynthesis in plants.

FIG. 12. shows a complementation test of SQS1 RNAi line of Arabidopsis resistance to nonhost pathogen P. syringae pv. tabaci. Squalene was supplied at the concentration as indicated above each panel when transplanting two-week old seedlings and thereafter watering. P. syringae pv. tabaci was infiltrated into leaves of four-week old plants at 1×10⁷ cfu/ml. The inoculated plants were kept at 19-21° C. and covered to keep high humidity. Pictures were taken 3 days post inoculation

FIG. 13. Transgenic Arabidopsis SQS1 RNAi lines, and the Arabidopsis mutants of smt2 and smt3. Pictures of symptoms of Arabidopsis infected by non-host pathogens P. syringae pv. tabaci (13A) and P. syringae pv. syringae (13B) were taken 5 days post inoculation. Four-week old Arabidopsis plants were inoculated with bacterial suspension at the concentration of 2×10⁷ cfu/ml by leaf infiltration. Bacterial growth (13C and 13D) was assessed 3 days post inoculation at 1×10⁶ cfu/ml.

FIG. 14. Stigmasterol is reduced in the smt2 (cvp) mutant and AtSQS1 RNAi lines (SSi2e and SSi5e) but significantly induced only by a nonhost pathogen. The content of sitosterol (FIG. 14A) and stigmasterol (FIG. 14B) in cvp mutant, AtSQS1 RNAi lines and wild type Arabidopsis were determined by GC-MS analysis before infection and 12 hours after infection with a nonhost pathogen P. syringae pv. tabaci at 105 cfu/ml. (FIG. 14C) The relative transcription of gene CYP710A1 encoding C22-sterol desaturase which specifically converts sitosterol to stigmasterol was measured by real-time PCR analysis of the total RNA extracted from Arabidopsis plant leaves infected with a nonhost pathogen P. syringae pv. tabaci, host pathogen P. syringae pv. tomato DC3000 and its hrcC negative mutant at 10⁵ cfu/ml at different time points. The leaf tissues collected at the same time for real-time PCR analysis were used to examine stigmasterol contents (FIG. 14D) by GC-MS analysis.

FIG. 15. A. Phenotypes of Arabidopsis AtSQS1 overexpression lines. B. Symptoms of Arabidopsis infected by host pathogens P. syringae pv. tomato DC3000 (B; left panel) and P. syringae pv. maculicola (B; right panel). Four-week old Arabidopsis plants were inoculated by directly dipping in bacterial suspension at the concentration of 1×10⁸ cfu/ml. Pictures were taken 3 days post inoculation. C.-D. Bacterial numbers in planta were determined by plating serial dilutions of leaf extracts three days after inoculation; C: P. syringae pv. tomato DC3000; D: P. syringae pv. Maculicola.

FIG. 16. Overexpression of SQS that confers disease tolerance in N. benthamiana. Five-week old N. benthamiana plants were inoculated with P. syringae pv. tabaci at 1×10⁴ cfu/ml by vacuum infiltration. (16A). The inoculated plants were kept at 20-22° C. The photo of disease symptoms was taken 5 days post inoculation. Bacterial growth (16B) was examined 3 and 5 days post inoculation respectively. Leaf disks were taken from four infiltrated plants at different time intervals, ground, and plated.

### BRIEF DESCRIPTION OF THE SEQUENCE LISTING

SEQ ID NO: 1 N. benthamiana SQS cDNA sequence

SEQ ID NO:2 N. benthamiana SQS DNA coding sequence

SEQ ID NO:3 A. thaliana SQS1 cDNA sequence

SEQ ID NO:4 A. thaliana SQS1 DNA coding sequence

SEQ ID NO:5 A. thaliana SQS1 DNA genomic sequence

SEQ ID NO:6 A. thaliana SQS2 cDNA sequence

SEQ ID NO:7 A. thaliana SQS2 DNA coding sequence

SEQ ID NO:8 A. thaliana SQS2 DNA genomic sequence

SEQ ID NO:9 A. thaliana SMT2 cDNA sequence

SEQ ID NO: 10 A. thaliana SMT2 DNA coding sequence

SEQ ID NO: 11 A. thaliana SMT2 DNA genomic sequence

SEQ ID NO: 12 N. benthamiana SQS polypeptide sequence

SEQ ID NO:13 A. thaliana SQS1 polypeptide sequence

SEQ ID NO: 14 A. thaliana SQS2 polypeptide sequence

SEQ ID NO: 15 A. thaliana SQS1 RNAi construct nucleotide sequence

SEQ ID NO: 16 primer AtSSiF

SEQ ID NO: 17 primer AtSSiR

SEQ ID NO:18 primer AtSSE1

SEQ ID NO: 19 primer AtSSE2

### DETAILED DESCRIPTION OF THE INVENTION

Nonhost resistance is the most common form of disease resistance exhibited by plants against the majority of potential pathogens in nature. Type I nonhost resistance does not produce any visible lesions whereas type II nonhost resistance results in a rapid hypersensitive response when pathogens are inoculated onto nonhost plants. Disease resistance is a desired trait in all commercially grown plants, thus new methods of combating disease by increasing plant resistance are of the utmost importance for agriculture. The current invention provides, in one embodiment, methods for modulating the expression of plant sterol biosynthesis pathways to yield an increase in plant disease resistance. In particular embodiments, genes in the sterol biosynthesis pathway are modulated, such as squalene synthase (SQS), a key enzyme catalyzing the first enzymatic step in sterol biosynthesis, and genes encoding sterol methyltransferase (a downstream enzyme in phytosterol biosynthesis). Overexpression of these genes and squalene synthase in particular in plants can increase resistance to nonhost pathogens, thereby reducing the need for other forms of disease control such as chemical or biological controls. Conversely, suppression of these genes can reduce resistance, which may be useful, for example, in instances where decreased disease resistance improves other aspect of the plant's phenotype and for characterizing the pathology of certain diseases or identifying other genes of interest.

The role of stigmasterol in plant disease resistance was also examined. These results suggest that stigmasterol also contributes to basal resistance and abiotic stress. Genetic engineering of plants to produce more stigmasterol may thus confer broad and durable resistance against pathogens.

### I. PLANT TRANSFORMATION CONSTRUCTS, NUCLEIC ACIDS AND POLYPEPTIDES

Certain embodiments of the current invention concern plant transformation constructs. For example, one aspect of the current invention provides a plant transformation vector comprising one or more squalene synthase coding sequence. Examples of such coding sequences for use with the invention encode the polypeptide of SEQ ID NO: 12-14, or the polypeptide encoded by any of SEQ ID NO:1-11. In certain embodiments of the invention, transformation constructs comprise the nucleic acid sequence of SEQ ID NO:1-11; and/or nucleic acid sequences encoding the polypeptides of SEQ ID NO: 12-14.

Coding sequences may be provided operably linked to a heterologous promoter, in either sense or antisense orientation. Expression constructs are also provided comprising these sequences, including antisense oligonucleotides thereof, as are plants and plant cells transformed with the sequences. The construction of vectors which may be employed in conjunction with plant transformation techniques using these or other sequences according to the invention will be known to those of skill of the art in light of the present disclosure (see, for example, Sambrook et al., 1989; Gelvin et al., 1990). The techniques of the current invention are thus not limited to any particular nucleic acid sequences.

Provided herein are also transformation vectors comprising nucleic acids capable of hybridizing to the nucleic acid sequences, for example, of SEQ ID NO: 1-11 and/or any sequence that encodes the polypeptide of nucleic acid sequences encoding the polypeptides of SEQ ID NO:12-14. As used herein, “hybridization,” “hybridizes” or “capable of hybridizing” is understood to mean the forming of a double or triple stranded molecule or a molecule with partial double or triple stranded nature. Such hybridization may take place under relatively high stringency conditions, including low salt and/or high temperature conditions, such as provided by a wash in about 0.02 M to about 0.15 M NaCl at temperatures of about 50° C. to about 70° C. for 10 min. In one embodiment of the invention, the conditions are 0.15 M NaCl and 70° C. Stringent conditions tolerate little mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

The invention provides a polynucleotide sequence identical over its entire length to each coding sequence set forth in the Sequence Listing. The invention also provides the coding sequence for the mature polypeptide or a fragment thereof, as well as the coding sequence for the mature polypeptide or a fragment thereof in a reading frame with other coding sequences, such as those encoding a leader or secretory sequence, a pre-, pro-, or prepro-protein sequence. The polynucleotide can also include non-coding sequences, including for example, but not limited to, non-coding 5′ and 3′ sequences, such as the transcribed, untranslated sequences, termination signals, ribosome binding sites, sequences that stabilize mRNA, introns, polyadenylation signals, and additional coding sequence that encodes additional amino acids. For example, a marker sequence can be included to facilitate the purification of the fused polypeptide. Polynucleotides of the present invention also include polynucleotides comprising a structural gene and the naturally associated sequences that control gene expression.

Another aspect of the present invention relates to the polypeptide sequences set forth in the Sequence Listing, as well as polypeptides and fragments thereof, particularly those polypeptides which exhibit squalene synthase activity and which have at least 85%, more preferably at least 90% identity, and most preferably at least 95% identity to a polypeptide sequence selected from the group of sequences set forth in the Sequence Listing, including 92%, 94%, 96%, 97%, 98% and 99% identity thereto.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J Applied Math, 48:1073 (1988).

Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)). The BLAST X program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well known Smith Waterman algorithm can also be used to determine identity.

Parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA 89:10915-10919 (1992); Gap Penalty: 12; and Gap Length Penalty: 4. A program which can be used with these parameters is publicly available as the “gap” program from Genetics Computer Group, Madison Wis. The above parameters along with no penalty for end gap may serve as default parameters for peptide comparisons.

Parameters for polynucleotide sequence comparison include the following: Algorithm: Needleman and Wunsch, J. Mol. Biol. 48:443-453 (1970); Comparison matrix: matches=+10; mismatches=0; Gap Penalty: 50; and Gap Length Penalty: 3. A program which can be used with these parameters is publicly available as the “gap” program from Genetics Computer Group, Madison Wis. The above parameters may serve as the default parameters for nucleic acid comparisons.

One beneficial use of the sequences provided by the invention will be in the alteration of plant phenotypes by genetic transformation with squalene synthase coding sequences. The squalene synthase coding sequence may be provided with other sequences and may be in sense or antisense orientation with respect to a promoter sequence. Where an expressible coding region that is not necessarily a marker coding region is employed in combination with a marker coding region, one may employ the separate coding regions on either the same or different DNA segments for transformation. In the latter case, the different vectors are delivered concurrently to recipient cells to maximize cotransformation.

The choice of any additional elements used in conjunction with an squalene synthase coding sequences will often depend on the purpose of the transformation. One of the major purposes of transformation of crop plants is to add commercially desirable, agronomically important traits to the plant, as described above.

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce genes corresponding to an entire biosynthetic pathway into a plant, such as all of the coding sequences for isoflavonoid biosynthesis.

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. In one embodiment of the invention, the native promoter of a squalene synthase coding sequence, or other sterol biosynthesis enzyme, is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is envisioned that squalene synthase coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue.

B. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of a squalene synthase coding sequence is used. Alternatively, a heterologous 3′ end may enhance the expression of sense or antisense squalene synthase coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### II. ANTISENSE AND RNAi CONSTRUCTS

Antisense and RNAi treatments represent one way of altering squalene synthase activity in accordance with the invention. In particular, constructs comprising a squalene synthase coding sequence, including fragments thereof, in sense and/or antisense orientation, may be used to decrease or effectively eliminate the expression of an squalene synthase in a plant. Alternatively, RNAi and antisense technology may be used to divert substrates to a selected pathway in the biosynthesis of phytosterols. The converse strategy could also be used. In this manner, the composition of phytosterols such as squalene in a plant may be selectively manipulated and beneficial phenotypes obtained.

Techniques for RNAi are well known in the art and are described in, for example, Lehner et al., (2004) and Downward (2004). The technique is based on the fact that double stranded RNA is capable of directing the degradation of messenger RNA with sequence complementary to one or the other strand (Fire et al., 1998). Therefore, by expression of a particular coding sequence in sense and antisense orientation, either as a fragment or longer portion of the corresponding coding sequence, the expression of that coding sequence can be down-regulated.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host plant or part thereof. In certain embodiments of the invention, such an RNAi or antisense oligonucleotide may comprise any unique portion of a nucleic acid sequence provided herein. In further embodiments of the invention, such a sequence comprises nucleic acids complementary to at least 18, 30, 50, 75 or 100 or more contiguous base pairs of the nucleic acid sequence of SEQ ID NOs:1-12 and/or any sequence that encodes the polypeptide of nucleic acid sequences encoding the polypeptides of SEQ ID NO:12-14, including the full length thereof.

Constructs may be designed that are complementary to all or part of the promoter and other control regions, exons, introns or even exon-intron boundaries of a gene. It is contemplated that the most effective constructs will include regions complementary to intron/exon splice junctions. Thus, it is proposed that a preferred embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., ribozyme; see above) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Virus-induced gene silencing (VIGS) is an RNA-mediated post-transcriptional gene silencing mechanism that may protect plants against foreign gene invasion. (Baulcombe 1999). VIGS can also be used as a tool for deciphering the function of genes in diverse plant species. Using VIGS analysis, a fragment of the plant gene of interest is directly inserted into a viral vector (reverse genetics approach) or an enriched cDNA library is cloned into the viral vector (fast-forward genetics approach; Baulcombe 1999). The target plant is then inoculated with the viral vector and the inserted gene is amplified by the viral replication system, spreading systemically in the infected plants, and resulting in the synthesis of dsRNA intermediates that trigger the RNA-mediated defense system (the RNA-induced silencing complex) for the degradation of the recombinant RNA and the corresponding host mRNA (Waterhouse et al. 2001; Baulcombe 2002). Among the several viral vector systems used to trigger VIGS, Tobacco rattle virus (TRV; contains bipartite positive-sense RNA genome RNA1 and RNA2; Matthews 1991)-derived vectors are preferred in dicots (Dinesh-Kumar et al. 2003; Lu et al. 2003a; Ryu et al. 2004). VIGS allows for analysis of genes that otherwise would produce lethal phenotypes when disrupted by conventional mutagenesis techniques; functional characterization of genes in different genetic backgrounds; and functional characterization of genes having redundant function within a gene family.

### III. GENETIC TRANSFORMATION

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

An illustrative embodiment of a method for delivering DNA into plant cells by acceleration is the Biolistics Particle Delivery System, which can be used to propel particles coated with DNA or cells through a screen, such as a stainless steel or NYTEX screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al. (1975) and MS media (Murashige and Skoog, 1962).

### IV. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by northern blotting. This technique will demonstrate the presence of an RNA species and give information about the integrity of that RNA. The presence or absence of an RNA species also can be determined using dot or slot blot northern hybridizations. These techniques are modifications of northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### V. BREEDING PLANTS OF THE INVENTION

In addition to direct transformation of a particular plant genotype with a construct prepared according to the current invention, transgenic plants may be made by crossing a plant having a selected DNA of the invention to a second plant lacking the construct. For example, a selected squalene synthase coding sequence can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. DEFINITIONS

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### VIGS-Mediated Forward Genetics Screen Identified Several cDNA Clones that when Silenced Compromised Nonhost Resistance

Forward genetic screens were performed using a N. benthamiana cDNA library (in recombinant TRV) as described (Anand et al., 2007). The silenced plants were challenged with P. syringae pv. tomato (strain T1) and P. syringae pv. glycinea. Virulence of these pathogens was verified by inoculating them on their hosts, tomato and soybean, respectively. Five days after inoculation, disease symptoms were seen on the inoculated host plants. P. syringae pv. glycinea is a type I nonhost pathogen for N. benthamiana and does not produce any symptoms upon inoculation. P. syringae pv. tomato is a type II nonhost pathogen for N. benthamiana and produces a host response (“HR”) upon inoculation.

After screening approximately 3,000 TRV-VIGS cDNA clones with replicates, approximately 50 cDNA clones were identified that when silenced compromised type I and/or type II nonhost resistance in N. benthamiana. These clones were then subject to secondary and tertiary screenings to remove false positives. Eleven non-redundant cDNA clones were ultimately identified that when silenced produced distinctive loss of nonhost resistance phenotypes in N. benthamiana (Table 1). The high number of initial false positives can be attributed to HR sensitivity to time and temperature. Subtle differences in timing of HR after inoculation may not be detected by visual observation. Therefore, a different method to screen for silenced plants that compromise nonhost resistance using fluorescence labeled bacteria was developed (Wang et al., 2007). Some of the clones shown in Table 1, when silenced, showed mild to severe stunted phenotype when compared to control (TRV::GFP inoculated plants). The combination of TRV infection and silencing of a gene important for plant development may have caused such severe phenotypes. It is therefore necessary to generate RNAi lines for further characterization.

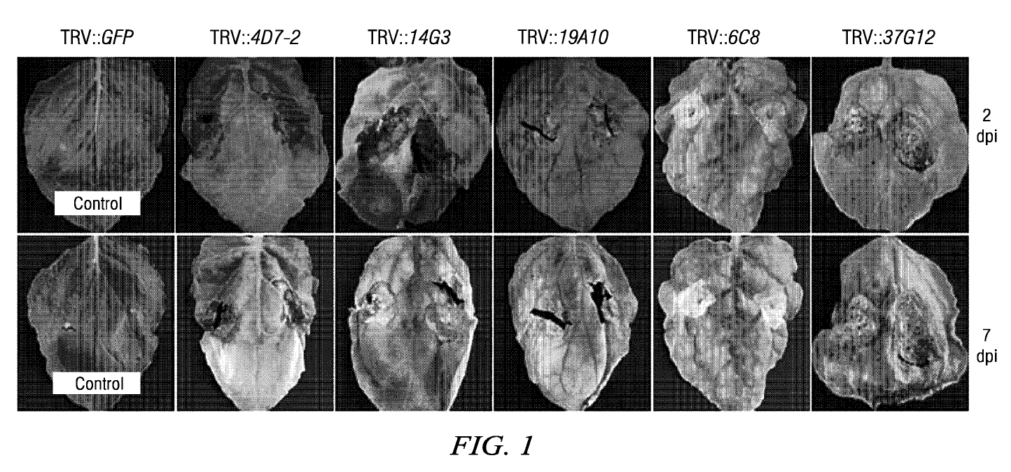

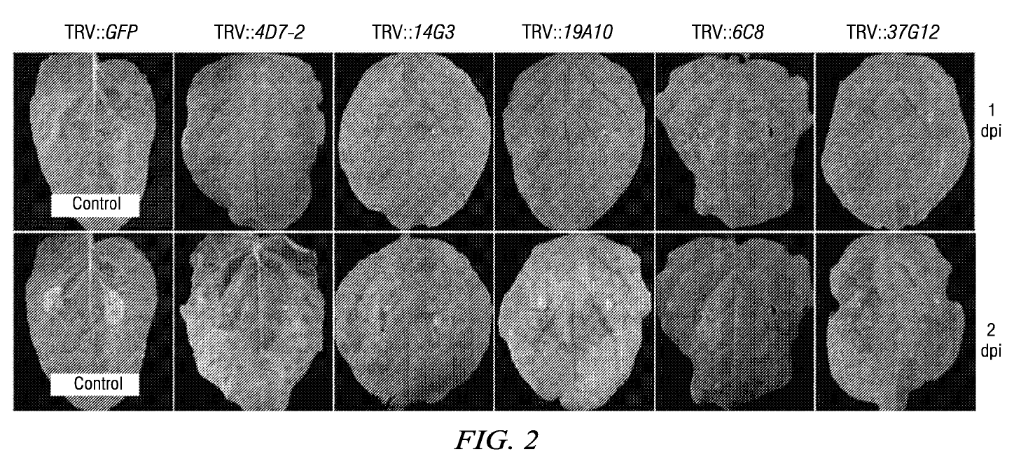

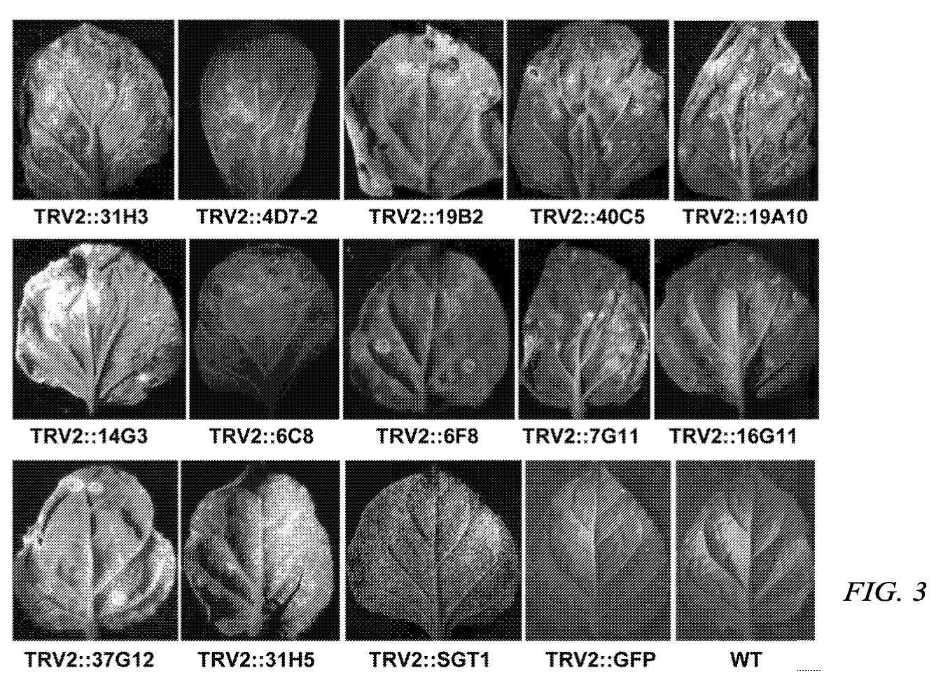

Eleven cDNA clones obtained from the screening were further characterized. These cDNA clones in TRV vector were PCR amplified using GATEWAY primers attB1 and attB2, and were subsequently sequenced. The sequences were subject to NCBI Blast search and the results are shown in Table 1. Agrobacteria containing these eleven cDNA clones in TRV vectors were individually used to silence the corresponding genes in N. benthamiana plants. A TRV vector containing a partial gene sequence of bacterial green fluorescent protein (GFP) was used as a control. N. benthamiana plants inoculated with TRV::GFP should not have any of its endogenous genes silenced because the GFP gene sequence has no homology with plant gene sequences. Three weeks after Agrobacterium inoculation, third and fourth leaves of the control and silenced plants were syringe infiltrated with either P. syringae pv. glycinea (type I nonhost pathogen) or P. syringae pv. tomato T1 (type II nonhost pathogen) at the concentration of 3×10⁷ cfu/ml. The control plants showed no symptoms upon infection with P. syringae pv. glycinea but showed a rapid HR upon infection with P. syringae pv. tomato (FIG. 1 and FIG. 2). Disease symptoms or a delay in HR development was observed in the silenced plants within 2-3 days after infection (FIG. 1 and FIG. 2). It is interesting to note that most of the cDNA clones compromised both type I and type II nonhost resistances when silenced in N. benthamiana. When GFPuv (e.g. Chalfie et al., 1994) labeled P. syringae pv. tomato T1 was used to inoculate plants at the concentration of 10⁵ cfu/ml by vacuum infiltration, no bacterial colonies (green spots) were observed in the control plant leaves by the naked eye under long wave length UV light (FIG. 3). However, strong green spots (bacterial accumulation) were clearly observed in the silenced plant leave within 4 days after inoculation (FIG. 3). Therefore, use of GFPuv labeled bacteria provides a rapid and accurate method for screening plant disease resistance in a large scale.

TABLE 1
Nb cDNA clones that compromise nonhost resistance when silenced.

| | Symptoms developed | Symptoms developed |
|---|---|---|
| | upon inoculation with | upon inoculation with |
| | P. syringae pv. | P. syringae pv. |
| cDNA clone (Blast results) | glycinea (Type I) | tomato (Type II) |
| Control (GFP) | None | HR |
| 4D7-2 (ABC transporter-like protein) | Disease | Slightly delayed HR |
| 6C8 (squalene Synthase) | Disease | Delayed HR |
| 6F8 (eukaryotic translation initiation | Disease | Delayed HR |
| factor 4A) | | |
| 7G11 (heat shock protein 70) | Disease | Disease |
| 14G3 (60S ribosomal protein L12) | Disease | Disease |
| 16G11 (glycolate oxidase) | Disease | Disease |
| 19A10 (unknown) | Disease | Delayed HR |
| 19A5 (60S ribosomal protein L19) | Disease | Delayed HR |
| 31H3 (putative GTP-binding protein) | Disease | Delayed HR |
| 31H5 (40S ribosomal protein S19) | Disease | None |
| 37G12 (60S ribosomal protein L9) | Disease | Disease |

*Disease = Progressive water soaked lesions

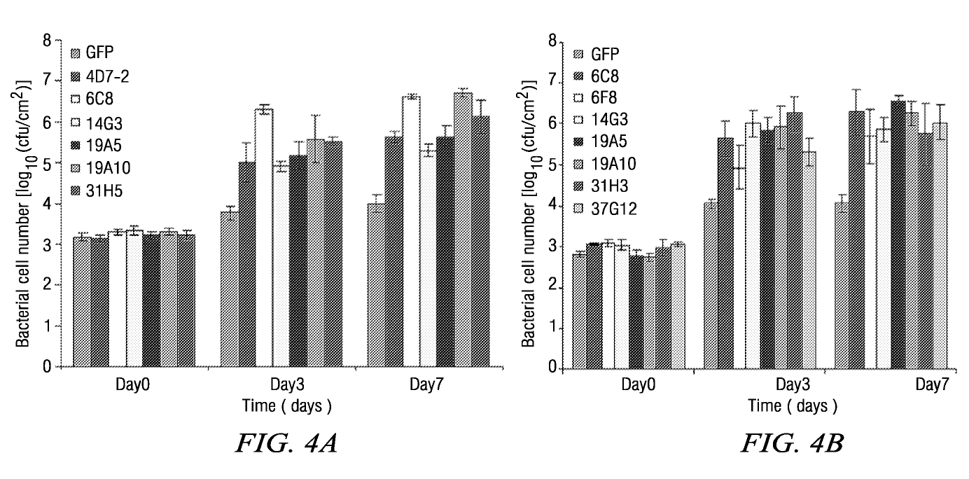

In a parallel experiment, the whole N. benthamiana control (TRV::GFP) and gene silenced plants were vacuum infiltrated with either P. syringae pv. glycinea or P. syringae pv. tomato at the concentration of 3×10⁴ cfu/ml, in order to achieve uniform infection. Leaf samples were collected at different times after infection and were subject to serial-dilution plating of bacteria to monitor the bacterial growth in planta in the silenced plants. As shown in FIG. 3 and FIG. 4, the cDNA silenced plants accumulated more bacteria at three to seven days after infection when compared to the control (GFP) plants. When these silenced plants were challenged with another type II nonhost pathogen, Xanthomonas campestris pv. vesicatoria, HR was delayed when compared to control. These results indicate that nonhost resistance of silenced plants is compromised against bacterial pathogens.

### Example 2

### N. benthamiana Squalene Synthase (NbSQS) is Required for Nonhost Resistance and Basal Resistance by Affecting Cell Membrane Leakage

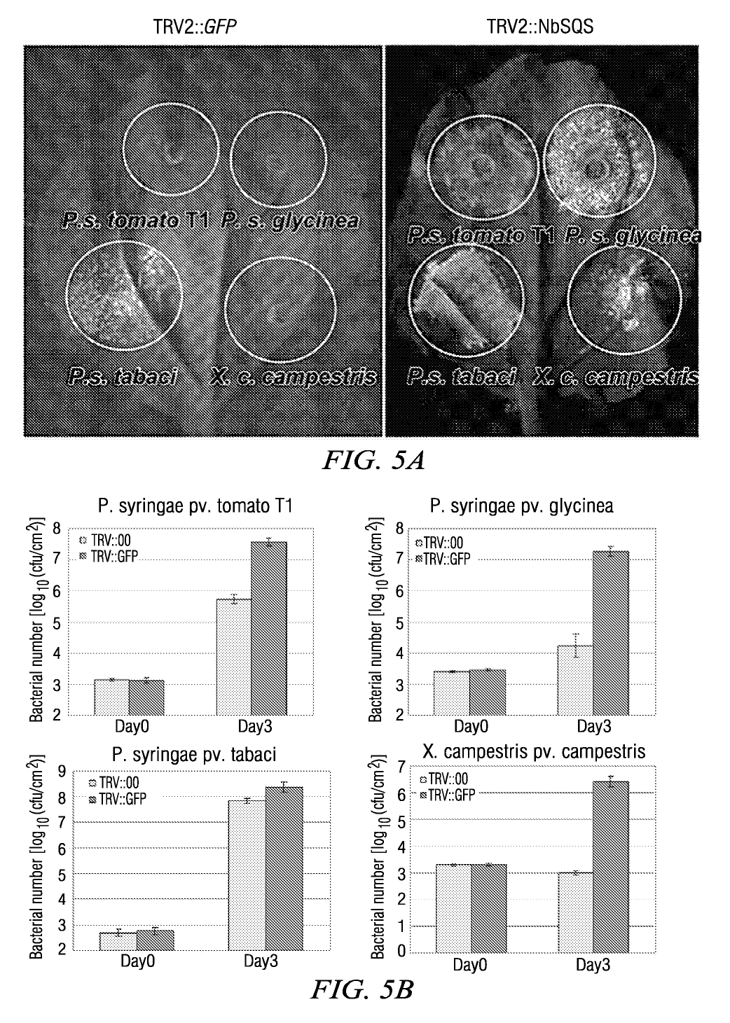

To examine whether NbSQS is required for resistance against host pathogen and other nonhost pathogens, other bacterial species Xanthomonas campestris pv. campestris labeled with GFPuv was used to inoculate NbSQS silenced N. benthamiana and control plants. Strong green fluorescent spots were observed in the NbSQS silenced plant leaves, but not in the control plants, indicating that silence of NbSQS supported the growth of nonhost pathogen X. campestris pv. campestris (FIG. 5). Furthermore, the NbSQS silenced plant leaf inoculated with host pathogen, GFPuv labeled P. syringae pv. tabaci, produced stronger green fluorescence than the control plant (FIG. 5). Examination of bacterial number in planta by traditional bacterial plating confirmed that the tested host and nonhost bacteria were significantly increased in the NbSQS silenced plants compared to the control plants (FIG. 5). Therefore, silence of NbSQS is not only compromised for nonhost resistance but also basal resistance.

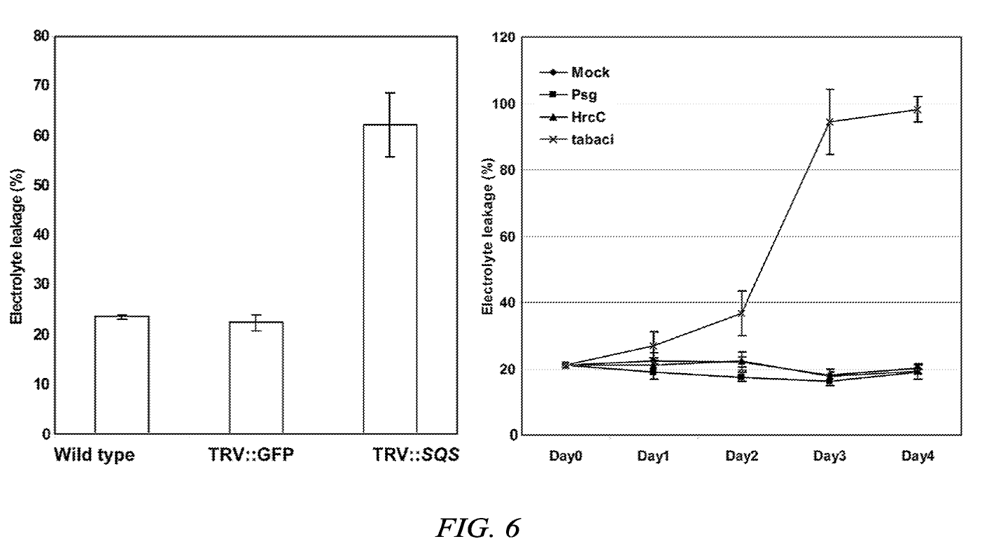

Squalene synthase is a key enzyme catalyzing the first enzymatic step in biosynthesis of phytosterols which are the major components of cell membrane. Silencing of SQS may affect biosynthesis of phytosterols, thus changing the structure of cell membrane. Therefore, silencing of NbSQS was examined, and found to result in cell membrane leakage by measuring the electrolyte conductivity of leaf discs. Surprisingly, NbSQS silenced plant leaves had more than 50% ion leakage, while the control plants had only around 20% ion leakage (FIG. 6). It has been hypothesized by plant pathologists that the development of plant foliage diseases comprises a process of cell leakage caused by plant pathogens, leading to final cell collapse. To examine this hypothesis and investigate the correlation between the cell leakage and pathogens, cell membrane leakage of wild type plants infected with different pathogens, including nonhost pathogen P. syringae pv. glycinea, host pathogen P. syringae pv. tabaci and its hrcC mutant which is deficient in type III (including Hrp-effector) secretion, was monitored. The results indicated that cell leakage was caused only by the host pathogen P. syringae pv. tabaci (FIG. 6). After two days inoculation, the plant leaves infected by P. syringae pv. tabaci had nearly 40% electrolyte leakage and reached almost 100% leakage by 3 days after inoculation. Infection with a P.s. tabaci hrcC mutant and a non-host pathogen, P. syringae pv. glycinea, did not increase electrolyte conductivity. This suggests that cell leakage resulted from suppression of the plant defense system by virulent pathogens injecting effector proteins into the plant cell.

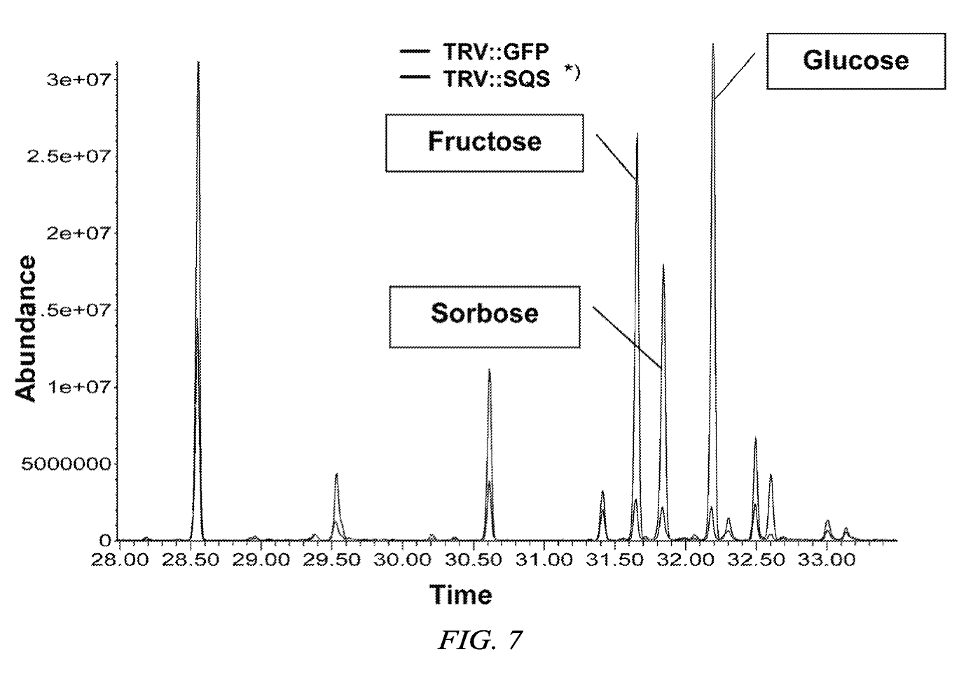

Since silencing of NbSQS in N. benthamiana plants resulted in ion leakage as indicated above, it might also lead to leakage of intracellular organic compounds. To examine this hypothesis, compounds in apoplastic fluids were examined by metabolite profiling using gas chromatography-mass spectrometry (GC-MS) (Broeckling et al., 2005). Apoplastic fluid was extracted by centrifugation of water-infiltrated plant leaves at low speed (˜500 g) and used for extraction of polar compounds. The extracts were subjected to analysis by GC-MS (FIG. 7). Surprisingly, there were 173±2.6 components detected in the apoplast from NbSQS silenced N. benthamiana, while only 123.7±4.9 from the control plants. Among the identified compounds, the levels of 20 compounds in the apoplastic fluid from NbSQS silenced N. benthamiana were at least 2 fold higher than that from the control plant (Table 2). Out of these 20 compounds, 10 compounds were sugars. These results indicate that silencing NbSQS in N. benthamiana leads to nutrient leakage.

TABLE 2
Comparative analysis of apoplastic fluids extracted
from the control and SQS silenced N. benthamiana

| Compounds | Ratio (SQS/Control) |
|---|---|
| Maltose | 10.37308 |
| Putrescine | 8.559926 |
| D-(+)-Glucose | 8.343614 |
| Glucose | 8.330549 |
| Ribose | 7.348089 |
| Xylitol | 6.867577 |
| Maltotriose | 6.467448 |
| Pyrrolidinone | 6.371822 |
| 2,3-Dihydroxybutanedioic acid | 6.184815 |
| Nicotine | 5.367337 |
| Propionic Acid, 2,3 Hydroxy | 5.180664 |
| D-(−)-Fructose | 4.982469 |
| Sorbose | 4.319295 |
| Xylose | 3.702044 |
| Erythritol | 3.257732 |
| Malonic Acid | 3.1365 |
| 1,6-Anhydroglucose | 2.985526 |
| Maleic Acid | 2.82817 |
| Glycerol | 1.968301 |

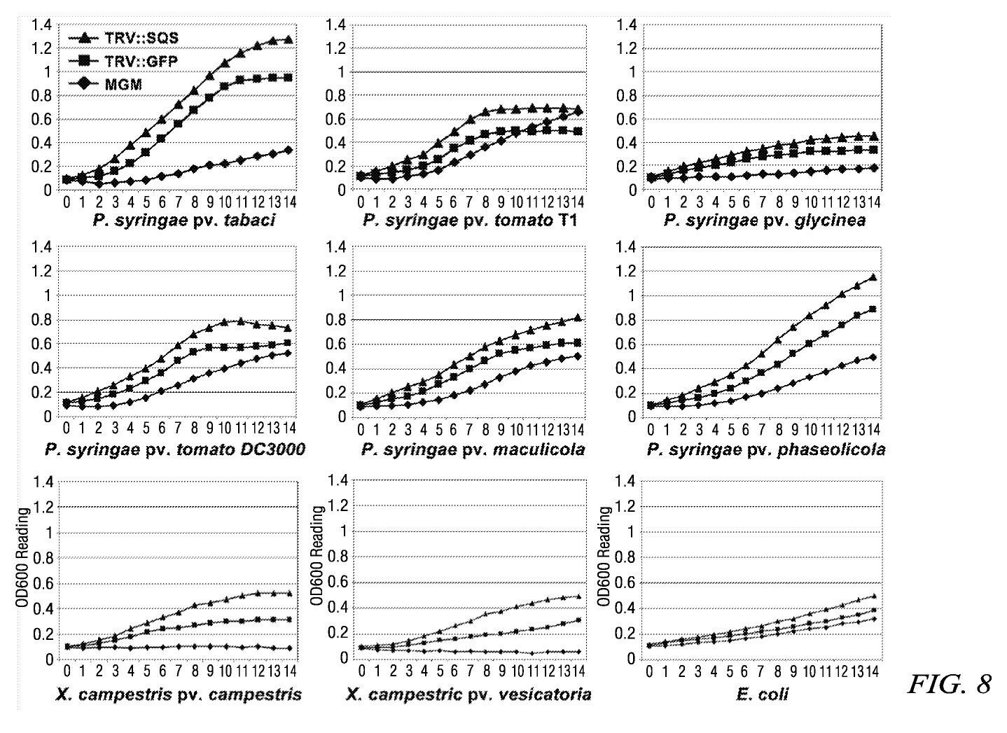

Plant pathogenic P. syringae strains primarily colonize in the apoplast and obtain nutrients directly from the apoplast for multiplication. It has been reported that the full strength apoplast extracts from tobacco and tomato supported the growth of host and nonhost pathogens as well as a non-pathogenic bacteria at similar growth rates (Rico and Preston, 2008). However, plants would be required to release more nutrients from cells into the apoplast if sufficient amounts of nutrients for were to be provided for phytopathogenic bacteria to multiply to high levels. To examine whether the high content of nutrients in the apoplast from the NbSQS silenced N. benthamiana plants support bacterial growth faster than that from control plant, the growth rates of different bacterial strains including host and nonhost phytopathogenic bacteria and nonpathogenic bacteria were measured using minimal growth medium (MGM) containing 5% of apoplast. The results indicated that all tested bacteria, including the non phytopathogenic E. coli, grew in MGM containing 5% apoplast from the NbSQS silenced N. benthamiana plants at a rate significantly faster than in that from control plants (FIG. 8). Interestingly, the nonhost pathogen P. syringae pv. tomato T1 stopped growth in MGM containing both apoplasts after 8 hours culture while it continuously grew in MGM without addition of apoplast and surpassed its growth in MGM with apoplast from control plant after 11 hours culture. This result suggests that an unknown substance in the apoplast inhibits the growth of P. syringae pv. tomato T1.

### Example 3

### Arabidopsis SQS RNAi Lines are Compromised for Nonhost and Basal Resistance

Genes encoding squalene synthase (NbSQS) that may play a role in nonhost resistance were identified by silencing NbSQS in N. benthamiana as described above and were further characterized by examining corresponding homologs in Arabidopsis. AtSQS has two gene family members: AtSQS1 and AtSQS2. AtSQS1 has a greater sequence homology to NbSQS (75.3% homology of amino acid sequences) than does AtSQS2 (68.7% homology of amino acid sequences). By searching the SALK and GABI T-DNA insertion databases, several Arabidopsis T-DNA knockout lines for AtSQS1 genes were identified. However, after many attempts, plants homozygous for AtSQS1 mutations could not be obtained. Most likely, these homozygous mutants are embryo lethal or sterile. Thus, RNAi experiments were utilized to lower the expression of the candidate genes, thus emphasizing the benefits of VIGS to study and assess function of genes for which homozygous mutants might be lethal in sexually propagated plants.

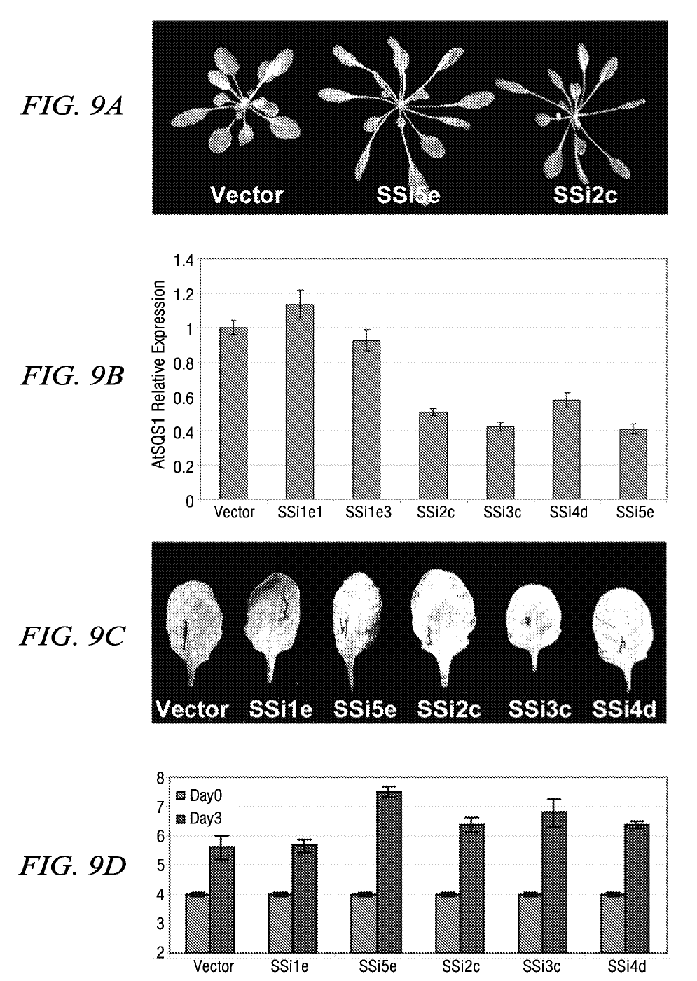

To make a construct for development of AtSQS1 RNAi lines, the partial AtSQS1 gene was amplified from Arabidopsis cDNA using the primers AtSSiF (5′-GGGGACAAGTTTGTACAAAAAAGCAGGCTGATTGAGAAAGCGGAGAAGCAGA-3′; SEQ ID NO: 16) and AtSSiR (5′-GGGGACCACTTTGTACAAGAAAGCTGGGTGCACAGAACCGAAATATGGAAGGA-3′; SEQ ID NO: 17). The full cDNA sequences of AtSQS1 and AtSQS2 are available (e.g. TIGR accessions At4g34640.1, At4g34650.1), and identified herein as SEQ ID NO:3 and SEQ ID NO:6. The amplified PCR product was cloned into a GATEWAY-ready binary vector pK7GWIWG2(I) (Karimi et al., 2002) under the control of CaMV 35S promoter by GATEWAY cloning technology following manufacture's instruction, resulting in vector pK7-AtSSi. Agrobacterium tumefaciens strain GV2260 containing pK7-AtSSi was used to transform Arabidopsis Col-0 using a floral dipping method (e.g. Clough and Bent, 1998). AtSQS1 RNAi lines had slightly slender leaves when compared to wild-type (FIG. 9A). The transcription of AtSQS1 in the RNAi lines was then determined by real-time PCR, showing that AtSQS1 was significantly reduced in 4 of 6 RNAi lines compared to the vector control (e.g. FIG. 9B). AtSQS1 RNAi lines were then challenged with a nonhost pathogen, P. syringae pv. tabaci, by leaf infiltration at 1×10⁶ cfu/ml. Disease symptoms were observed in the transgenic AtSQS1 Arabidopsis RNAi lines showing significantly reduced transcription of AtSQS1, but not in the empty vector control line or in the RNAi line (SSi1e) that did not display a reduced AtSQS1 transcript level (FIG. 9C). Correspondingly, bacterial growth in planta was examined by plating serial dilutions of ground leaf samples on KB medium with appropriate antibiotics. The results confirmed that the transgenic AtSQS1 RNAi lines of Arabidopsis were susceptible to nonhost pathogen P. syringae pv. tabaci when compared to empty vector control Arabidopsis. (FIG. 9D). The sequence of the RNAi fragment for AtSQS1 is given as SEQ ID NO: 15.

### Example 4

### The Role of Squalene Synthase (SQS) in Nonhost Resistance

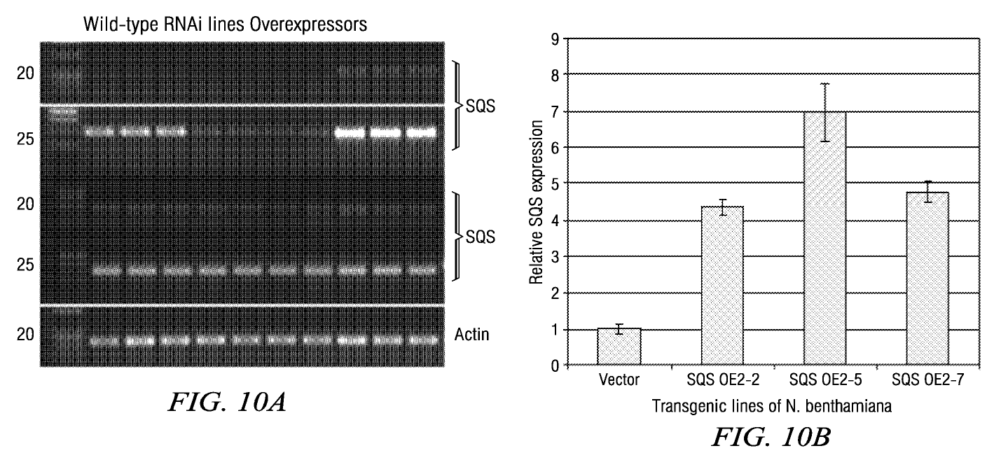

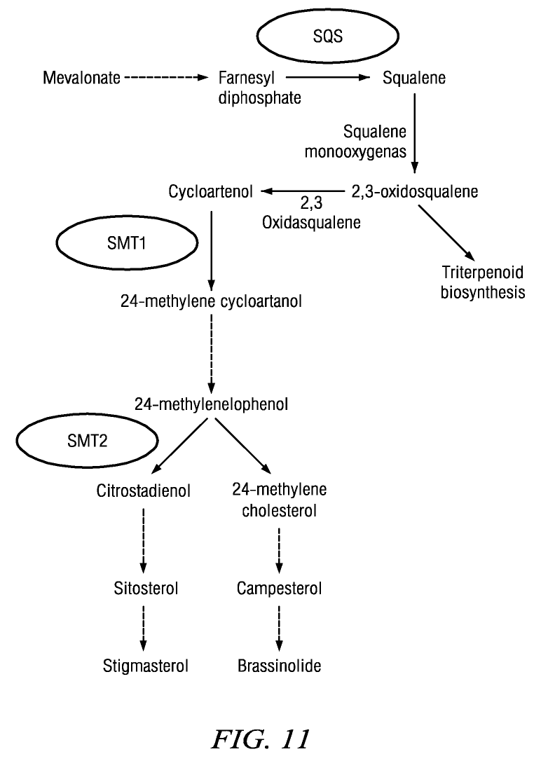

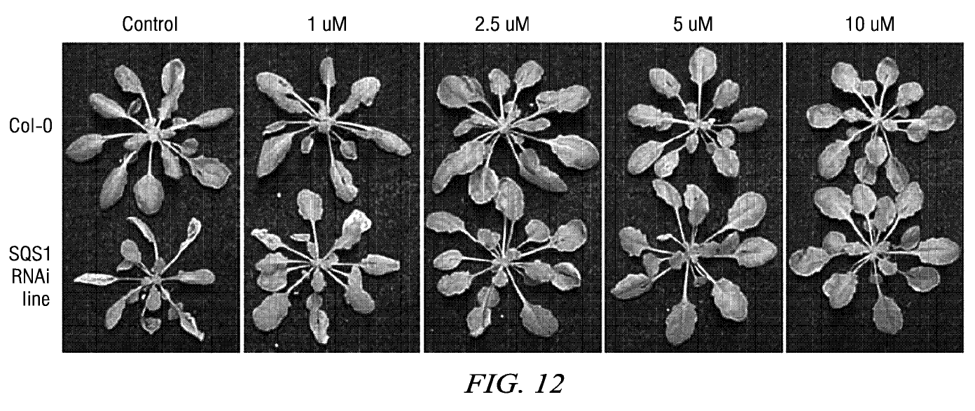

It was hypothesized that SQS1 RNAi lines would have less squalene compared to wild-type plants. Arabidopsis has two SQS genes: SQS1 and SQS2. T-DNA knockout lines for SQS2 are not available in SALK or GABI collections. Even though both SQS1 and SQS2 have more than 80% nucleotide identity, in the SQS1 RNAi line, only the transcripts of SQS1 was reduced but not SQS2 (FIG. 10). A brief pathway of sterol biosynthesis in plants and also other pathways that may be affected by silencing of SQS1 are shown in FIG. 11. To show that squalene is required for nonhost resistance, SQS RNAi lines were fed with various different concentrations of squalene (1 μM to 10 μM). SQS1 RNAi lines treated with squalene recovered nonhost resistance in a dose dependent manner and 10 μM squalene completely recovered resistance phenotype back to wild-type levels (FIG. 12). It was therefore shown that squalene plays a critical role in conferring nonhost resistance against certain pathogens, and the role is recoverable in susceptible RNAi lines by squalene dosing.

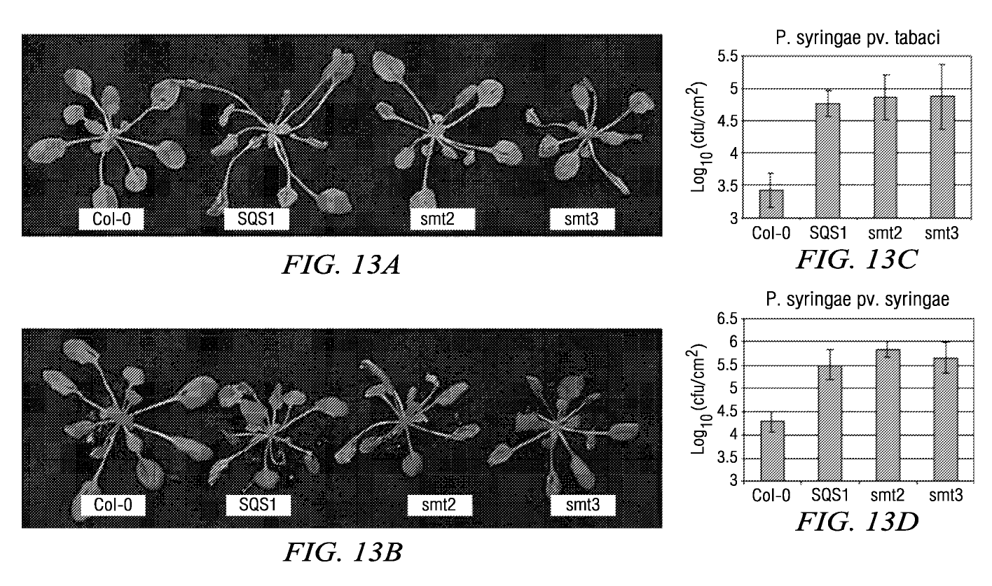

As shown in FIG. 11, squalene can affect multiple pathways, and different pathways required for nonhost resistance were identified. A mutant of sterol methyl transferase 2 (SMT2) was studied for susceptibility to nonhost resistance. An Arabidopsis smt2 mutant, also called as cotyledon vascular pattern 1 (cvp1) mutant has already been identified (Carland et al. 2002). Seeds were obtained of cvp1 mutants (cvp1-4) and challenged cvp1-4 plants with nonhost pathogens P. syringae pv. tabaci and P. syringae pv. syringae. Strikingly, cvp1-4 plants were susceptible, to the same degree as SQS1 RNAi lines, to both of the nonhost pathogens tested (FIG. 13). An Arabidopsis T-DNA knockout of SMT3 was also obtained and smt3 is also susceptible to nonhost pathogens. (FIG. 13). These results also indicate that sterols are required for nonhost resistance against certain pathogens.

### Example 5

### SQS-Involved Biosynthesis of Stigmasterol Plays an Important Role in Plant Disease Resistance

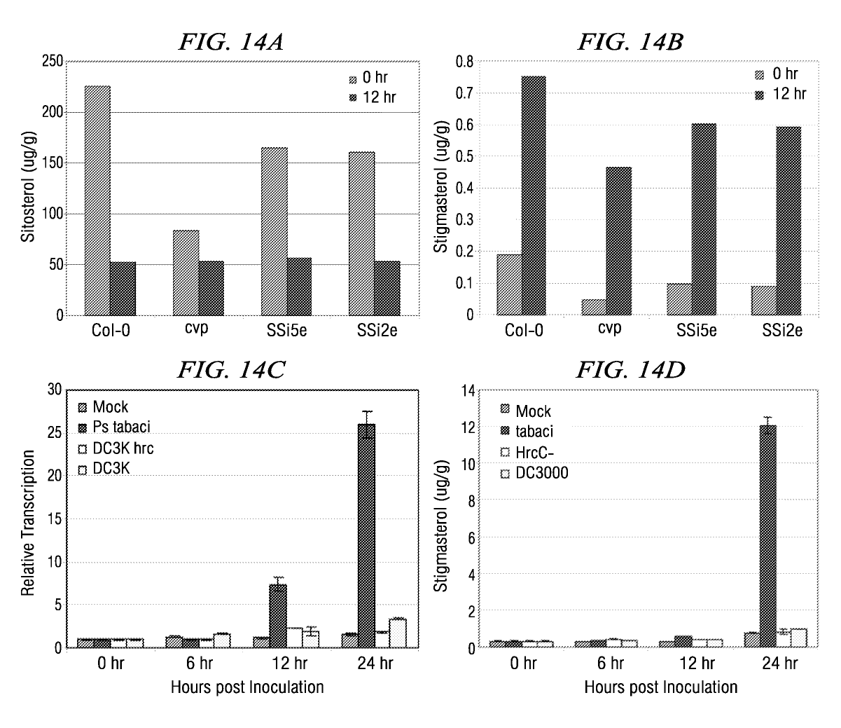

Squalene synthase catalyzes the first step in biosynthesis of various phytosterols, while sterol methyltransferase is a key enzyme leading to metabolite pathway for the products of sitosterol and stigmasterol. Therefore, Arabidopsis smt2 mutant and AtSQS1 RNAi lines may contain less sitosterol and stigmasterol which may be related to plant disease resistance, as phytosterols not only modulate membrane permeability and fluidity, but also may serve as signal transduction molecules (Borner et al., 2005). To test this hypothesis, the content of sitosterol and stigmasterol was first examined in an Arabidopsis smt2 mutant and in AtSQS1 RNAi lines. The frozen plant leaf tissue in liquid nitrogen was lyophilized and used for extraction of phytosterols using choloroform/methanol (Morikawa et al., 2006). The total sterols were analyzed by GC-MS. As expected, sitosterol and stigmasterol in the smt2 mutant and AtSQS1 RNAi lines were significantly reduced when compared to the wild type Arabidopsis (FIG. 14). Then the plants were inoculated with a nonhost pathogen P. syringae pv. tabaci at 10⁵ cfu/ml by vacuum infiltration. Significantly reduction of sitosterol but dramatic induction of stigmasterol was observed in all Arabidopsis lines 12 hours after inoculation (FIG. 14). However, induction levels of stigmasterol in smt2 mutant and AtSQS1 RNAi lines were lower than that in the wild-type Arabidopsis, indicating that stigmasterol plays important role in plant nonhost resistance. To further investigate the role of stigmasterol in plant disease resistance, wild-type Arabidopsis were inoculated with different pathogens including a nonhost pathogen P. syringae pv. tabaci, a host pathogen P. syringae pv. tomato DC3000 and its hrcC mutant. In addition to measurement of stigmasterol, expression of the gene CPY710A1 encoding encoding C22-sterol desaturase which specifically converts sitosterol to stigmasterol was also examined by real-time PCR analysis of the total RNA extracted from Arabidopsis plant leaves. Surprisingly, the transcript level of gene CPY710A1 was dramatically increased 12 hours after inoculation with a nonhost pathogen and reached more than 25 fold than control at the 24 hpi (FIG. 14). Correspondingly, the level of stigmasterol increased more than 40 fold after 24 hours infection with P. syringae pv. tabaci. A slight increase of the gene CYP710A1 and stigmasterol was also observed after infection with the host pathogen and in the hrcC mutant as well as mock treatment, while the increases after pathogen infection were slightly higher when compared to mock treatment. These results suggest that stigmasterol also contributes to basal resistance and abiotic stress, although it is primarily related to plant nonhost resistance. Genetic engineering of plants to produce more stigmasterol may thus confer broad and durable resistance against pathogens.

### Example 6

### Overexpression of SQS Conferred Disease Tolerance in Arabidopsis and N. Benthamiana

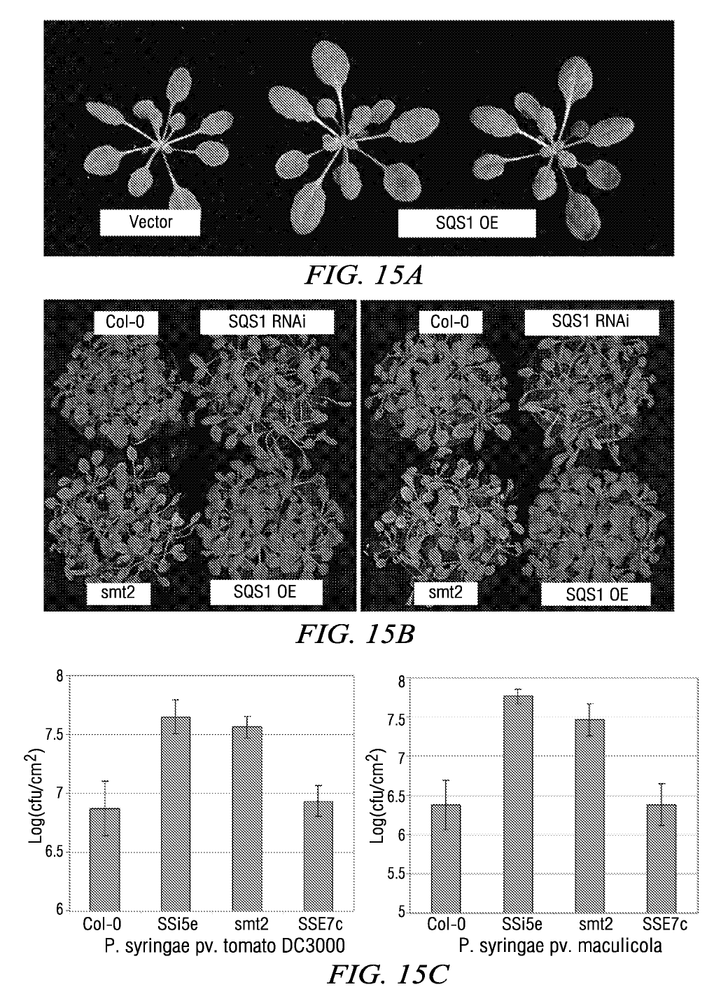

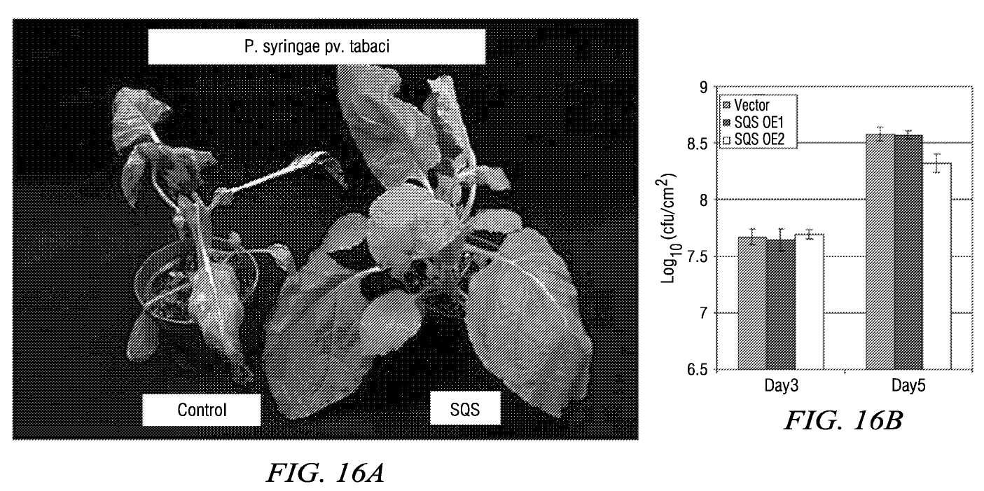

The full length gene of SQS1 from Arabidopsis (SEQ ID NO: 4) was amplified using the primer pair AtSSE1 (5′-GGGGACAAGTTTGTACAAAAAAGCAGGCTTAGAAGGAGATAGAACCATGGGGAGC TTGGGGACGAT-3′; SEQ ID NO:18) and AtSSE2 (5′-GGGGACCACTTTGTACAAGAAAGCTGGGTCTCAGTTTGCTCTGAGATATGCAAAG-3′; SEQ ID NO:19) and cloned into a binary vector pMDC32 (Curtis and Grossniklaus, 2003) under the control of double CaMV 35S promoter using GATEWAY cloning technology as described above, resulting a construct 35S:AtSSE. Several transgenic Arabidopsis lines that overexpress SQS1 were generated using floral dipping transformation (FIG. 15). Surprisingly, these lines were more tolerant to disease symptoms when inoculated with Arabidopsis pathogens P. syringae pv. tomato DC3000 and P. syringae pv. maculicola (FIG. 15). The full length SQS gene was amplified from N. benthamiana by RACE PCR and cloned into a binary vector pMDC32 under the control of double CAMV 35 S promoter resulting construct 35S:NbSSE. The transformation of N. benthamiana with a construct comprising SEQ ID NO: 2 was conducted using the modified procedure of Horsch's method (Horsch et al., 1985). The transcripts of NbSQS were significantly higher in these transgenic lines (FIG. 10). When challenged with a pathogen, P. syringae pv. tabaci, these transgenic lines were tolerant to disease symptoms, although the amount of bacterial growth in the overexpressor was not significantly less than the control (FIG. 16). These results indicate that overexpression of SQS increases tolerance to disease symptoms.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references listed below are incorporated herein by reference to the extent that they supplement, explain, provide a background for, or teach methodology, techniques, and/or compositions employed herein.
U.S. Pat. No. 4,535,060
U.S. Pat. No. 5,302,523
U.S. Pat. No. 5,322,783
U.S. Pat. No. 5,384,253
U.S. Pat. No. 5,464,765
U.S. Pat. No. 5,508,184
U.S. Pat. No. 5,538,877
U.S. Pat. No. 5,538,880
U.S. Pat. No. 5,545,818
U.S. Pat. No. 5,550,318
U.S. Pat. No. 5,563,055
U.S. Pat. No. 5,591,616
U.S. Pat. No. 5,610,042
Abdullah et al., Biotechnology, 4:1087, 1986.
Adlercreutz and Mazur, Ann. Med. 29:95-120, 1997.
Akashi et al., Biochem. Biophys. Res. Comm. 251:67-70, 1998.
Akashi et al., Plant Cell Physiol. 44:103-112, 2003.
Anand, et al., Molecular Plant-Microbe Interactions 20: 41-52, 2007.
Bates, Mol. Biotechnol., 2(2):135-145, 1994.
Battraw and Hall, Theor. App. Genet., 82(2):161-168, 1991.
Baulcombe, D. C. Curr. Opin. Plant Biol. 2:109-113, 1999.
Baulcombe, D. C., Curr. Biol. 12:R82., 2002.
Bevan et al., Nucleic Acids Research, 11(2):369-385, 1983.
Bhandari et al., J. Chem. Soc. Perkin Trans., 839-849, 1992.
Bhattacharjee; An; Gupta, J. Plant Bioch. and Biotech. 6, (2):69-73. 1997.
Borner et al., Plant Physiol. 137: 104-116, 2005.
Bower et al., The Plant Journal, 2:409-416, 1992.
Broeckling et al., J. Exp. Bot. 56: (410):323-336, 2005
Buising and Benbow, Mol Gen Genet, 243(1):71-81. 1994.
Callis, Fromm, Walbot, Genes Dev., 1:1183-1200, 1987.
Carland F M, et al. Plant Cell 14: 2045-2058, 2002
Casa et al., Proc. Nat'l Acad. Sci. USA, 90(23):11212-11216, 1993.
Chalfie et al., Science 263:802-805, 1994.
Chandler et al., Plant Cell, 1:1175-1183, 1989.
Choudhary et al., Plant Cell Rep., 9:42-46, 1990.
Chu et al., Scientia Sinica, 18:659-668, 1975.
Church and Gilbert, Proc. Natl. Acad. Sci. USA, 81:65-71, 1984.
Clemens and Barz, Phytochemistry, 41:457-460, 1996.
Clemens et al., Phytochemistry, 32:653-657, 1993.
Clough and Bent, Plant J., 16:735-43, 1998.
Conkling et al., Plant Physiol., 93:1203-1211, 1990.
Curtis and Grossniklaus, Plant Physiol. 133:462-469, 2003.
Dalkin et al., Plant Physiol., 92:440-446, 1990.
DE 3642 829
De Block et al., The EMBO Journal, 6(9):2513-2518, 1987.
De Block, De Brouwer, Tenning, Plant Physiol., 91:694-701, 1989.
Dellaporta et al., In: Chromosome Structure and Function: Impact of New Concepts, 18th Stadler Genetics Symposium, 11:263-282, 1988.
Dinesh-Kumar, S. P., et al., In: Plant Functional Genomics., Grotewold, E., (Ed.). Vol. 236. Totowa: Humana Press, 287-293, 2003.
Dewick and Martin, Phytochemistry, 18:591-596, 1979.
Dewick and Ward, Phytochemistry, 17:1751-1754, 1978.
Dewick, In: The Flavonoids: Advances in Research Since 1986., Harborne (Ed.), London: Chapman and Hall, 117-238, 1993.
Dixon and Ferreira, Phytochemistry, 60:205-211, 2002.
Dixon and Sumner, Plant Physiol., 131:878-885, 2003.
Dixon et al., Mol. Plant. Pathol., 3:371-390, 2002.
Dixon, In: Comprehensive Natural Products Chemistry, Vol. 1, Sankawa (Ed.), Elsevier, Oxford, 773-823, 1999.
Drewes et al., Phytochemistry. 59:739-747, 2002.
Ebert et al., 84:5745-5749, Proc. Nat'l Acad. Sci. USA, 1987.
EPA App. 154,204, 1985
Flor, Annu. Rev. Phytopathol. 9: 275-296, 1971.
Fraley et al., Bio/Technology, 3:629-635, 1985.
Fromm et al., Nature, 319:791-793, 1986.
Fulton D C, et al, Phytochemistry 34: 139-145, 1993.
Gallie et al., Plant Cell, 1:301-311, 1989.
Gelvin et al., In: Plant Molecular Biology Manual, 1990.
Ghosh-Biswas et al., J. Biotechnol., 32(1):1-10, 1994.
Gietz et al., Nucleic Acid Research, 20:1425, 1992.
Gunia et al., Z. Naturforsch. 46:58-66, 1991.
Hagio, Blowers, Earle, Plant Cell Rep., 10(5):260-264, 1991.
Hammond-Kosack and Jones, Ann. Rev. Plant Physiol. Plant Mol. Biol. 48: 2-45, 1997
Haseloff et al., Proc. Natl. Acad. Sci. USA, 94(6):2122-2127, 1997.
He and Dixon, Plant Cell, 12:1689-1702, 2000.
He et al., Plant Cell Reports, 14 (2-3):192-196, 1994.
Heath, M. Curr. Opin. Plant. Biol. 3, 315-319, 2000.
Hensgens et al., Plant Mol. Biol., 22(6):1101-1127, 1993.
Hiei et al., Plant. Mol. Biol., 35(1-2):205-218, 1997.
Higgins, Physiol. Plant Pathol., 2:289-300, 1972.
Hinchee et al., Bio/technol., 6:915-922, 1988.
Hinderer et al., FEBS Lett. 214:101-106, 1987.
Horsch R B, et al., Science, 227:1229-1231, 1985.
Hudspeth and Grula, Plant Mol. Biol., 12:579-589, 1989.
Ikuta et al., Bio/technol., 8:241-242, 1990.
ILDIS (International Legume Database and Information Service), In: Phytochemical Dictionary of the Leguminosae. Vol. 1, Bisby (Eds.), London: Chapman and Hall, 1994.
Ingham, In: Phytoalexins, Bailey and Mansfield, (Eds.), New York: Halstead Press, 21-80, 1982.
Ishida et al., Nat. Biotechnol., 14(6):745-750, 1996.
Kaeppler et al., Plant Cell Reports 9: 415-418, 1990.
Kaeppler, Somers, Rines, Cockburn, Theor. Appl. Genet., 84(5-6):560-566, 1992.
Karimi M, et. al., Trends Plant Sci. 7:193-195, 2002.
Katz et al., J. Gen. Microbiol., 129:2703-2714, 1983.
Klee, Yanofsky, Nester, Bio-Technology, 3(7):637-642, 1985.
Knittel, Gruber; Hahne; Lenee, Plant Cell Reports, 14(2-3):81-86, 1994.
Kochs and Grisebach, Eur. J. Biochem., 155:311-318, 1986.
Kochs and Grisebach, Arch. Biochem. Biophys., 273:543-553, 1989.
Lambert et al., Phytochemistry, 34:1515-1520, 1993.
LatundeDada et al., J. Biol. Chem., 276:1688-1695, 2001.
Lawton et al., Plant Mol. Biol. 9:315-324, 1987.
Lazzeri, Methods Mol. Biol., 49:95-106, 1995.
Liu and Dixon, Plant Cell, 13:2643-2658, 2001.
Liu et al., Proc. Natl. Acad. Sci. USA, 99:14578-14583, 2002.
Lu, R., et al., EMBO J. 22:5690-5699, 2003.
Mabry et al., In: The systematic identification of the flavonoids. New York: Springer-Verlag, 1970.
Mackenbrock and Barz, Z. Naturforsch. 38:708-710, 1983.
Mackenbrock et al., J. Plant Physiol., 142:385-391, 1993.
Martin G B, Curr. Opin. Plant Biol. 2: 273-279, 1999.
McCabe, Martinell, Bio-Technology, 11(5):596-598, 1993.
McCormac et al., Euphytica, v. 99 (1) p. 17-25: 1998.
Mihaliak et al., Meth. Plant Biochem., 9:261-279, 1993.
Morikawa et al., Plant Cell, 18:1008-1022, 2006.
Murakami et al., Mol. Gen. Genet., 205:42-50, 1986.
Murashige and Skoog, Physiol. Plant., 15:473-497, 1962.
Nagatani et al., Biotech. Tech., 11(7):471-473, 1997.
Nicholas et al., Phytochemistry, 24:2881-2883, 1985.
Odell et al., Nature, 313:810-812, 1985.
Ogawa et al., Sci. Rep., 13:42-48, 1973.
Olah and Sherwood, Phytopathology, 61:65-69, 1971.
Omirulleh et al., Plant Mol. Biol., 21(3):415-428, 1993.
Overkamp et al., Plant Sci., 155:101-108, 2000.
Ow et al., Science, 234:856-859, 1986.
PCT App. WO 92/17598
PCT App. WO 94/09699
PCT App. WO 95/06128
PCT App. WO 97/04103
PCT App. WO 97/41228
Pompon et al., Meth. Enzymol., 272:51-64, 1996.
Potrykus et al., Mol. Gen. Genet., 199:183-188, 1985.
Prasher et al., Biochem. Biophys. Res. Commun., 126(3):1259-1268, 1985.
Reichel et al., Proc. Natl. Acad. Sci. USA, 93 (12) p. 5888-5893. 1996.
Restrepo et al., Plant Cell, 2:987-998, 1990.
Rico et al., Mol. Plant-Microbe Interact., 21(2): 269-282, 2008.
Ritala et al., Plant Mol. Biol., 24(2):317-325, 1994.
Rogers et al., Methods Enzymol., 153:253-277, 1987.
Ryu, C.-M., et al., Plant J. 40:322-331, 2002.
Sambrook et al., In: Molecular Cloning—A Laboratory Manual (second edition), Cold Spring Harbour Laboratory Press, 1989.
Schumacher et al., Plant Cell Rep., 6:410-413, 1987.
Sheen et al., Plant Journal, 8(5):777-784, 1995.
Shimada et al., Plant Sci., 160:37-47, 2000.
Simmonds and Stevenson, J. Chem. Ecol., 27:965-977, 2001.
Singsit et al., Transgenic Res., 6(2):169-176, 1997.
Stalker et al., Science, 242:419-422, 1988.
Sullivan et al., Mol. Gen. Genet., 215(3):431-440, 1989.
Sutcliffe, Proc. Natl. Acad. Sci. USA, 75:3737-3741, 1978.
Tebayashi et al., J. Exp. Bot., 52:681-668, 2001.
Thillet et al., J. Biol. Chem., 263:12500-12508, 1988.
Thomas et al., Plant Sci. 69:189-198, 1990.
Thompson et al., Euphytica, 85(1-3):75-80, 1995.
Thompson et al., The EMBO Journal, 6(9):2519-2523, 1987.
Threlfall and Whitehead, Phytochemistry 27: 2567-2580, 1988.
Tian, Sequin, Charest, Plant Cell Rep., 16:267-271, 1997.
Tingay et al., The Plant Journal v. 11 (6) p. 1369-1376. 1997.
Tolleson et al., J. Agric. Food Chem., 50:4783-4790, 2002.
Tomes et al., Plant. Mol. Biol. 14(2):261-268, 1990.
Torbet, Rines, Somers, Crop Science, 38(1):226-231, 1998.
Torbet, Rines, Somers, Plant Cell Reports, 14(10):635-640, 1995.
Toriyama et al., Theor Appl. Genet., 73:16, 1986.
Tsukada; Kusano; Kitagawa, Plant Cell Physiol., 30(4)599-604, 1989.
Twell et al., Plant Physiol 91:1270-1274, 1989.
Vogeli and Chappell, Plant Physiol. 115: 1291-1296, 1988
Van Eck; Blowers; Earle, Plant Cell Reports, 14(5):299-304, 1995.
Vasil et al., Plant Physiol., 91:1575-1579, 1989.
Walker et al., Proc. Natl. Acad. Sci. USA, 84:6624-6628, 1987.
Wang et al., Molecular and Cellular Biology, 12(8):3399-3406, 1992.
Wang, K, et al. New Phytologist 174: 212-223, 2007.
Waterhouse, et al., Nature 411:834-842, 2001.
Wong, In: The Flavonoids. Part 2, Harborne et al. (Eds.), New York, San Francisco: Academic Press, 743-800, 1975.
Yamada et al., Plant Cell Rep., 4:85, 1986.
Yang and Russell, Proc. Natl. Acad. Sci. USA, 87:4144-4148, 1990.
Zheng and Edwards, J. Gen. Virol., 71:1865-1868, 1990.
Zook and Kuc, Physiol. Mol. Plant Pathol. 39: 377-390, 1991.
Zukowsky et al., Proc. Natl. Acad. Sci. USA, 80:1101-1105, 1983.

What is claimed is:

1. A method for increasing disease resistance in a plant comprising expressing in the plant a heterologous nucleic acid encoding squalene synthase or sterol methyltransferase.

2. The method of claim 1 comprising expressing in the plant a nucleic acid encoding squalene synthase.

3. The method of claim 1, wherein the nucleic acid sequence is operably linked to a promoter functional in a plant cell and wherein the nucleic acid sequence is selected from the group consisting of:
(a) a nucleic acid sequence comprising SEQ ID NO: 1, SEQ ID NO:2, SEQ ID NO:3, SEQ ID NO:4, SEQ ID NO:5 or SEQ ID NO:6, SEQ ID NO:7, SEQ ID NO:8, SEQ ID NO:9, SEQ ID NO:10, or SEQ ID NO:11;
(b) a nucleic acid sequence encoding the polypeptide of SEQ ID NO: 12, SEQ ID NO: 13, or SEQ ID NO: 14;
(c) a nucleic acid sequence having at least 85% sequence identity to the nucleic acid sequence of (a) or (b);
(d) a nucleic acid sequence complementary to the nucleic acid sequence of (a) or (b); and
(e) a nucleic acid sequence that hybridizes to the sequence of (a) or (b) under conditions of 0.15 M NaCl at 70° C.

4. The method of claim 3, wherein the plant is a monocot.

5. The method of claim 3 wherein the plant is a dicot.

6. The method of claim 5, wherein the plant is Arabidopsis thaliana or Nicotiana benthamiana.

7. The method of claim 3, wherein the promoter is inducible, organelle-specific, tissue-specific, pathogen-induced, cell-specific, developmentally-specific or constitutive.

8. The method of claim 3, wherein the construct comprises at least one additional sequence chosen from the group consisting of: a regulatory sequence, a selectable marker, a screenable marker, a secretable marker, a leader sequence, and a terminator.

9. The method of claim 3, wherein the construct is inherited from a parent plant of the plant.

10. The method of claim 3, wherein the plant is an R₀ transgenic plant.

11. The method of claim 3 wherein resistance to P. syringae pv. glycinea or P. syringae pv. tomato is increased.

12. A method of producing a transgenic plant having enhanced disease resistance comprising introducing into the plant a recombinant DNA construct comprising a promoter functional in plants operably linked to a nucleic acid molecule selected from the group consisting of:
(a) a nucleic acid sequence comprising the sequence of SEQ ID NO:1, SEQ ID NO:2, SEQ ID NO:3, SEQ ID NO:4, SEQ ID NO:5 or SEQ ID NO:6, SEQ ID NO:7, SEQ ID NO:8, SEQ ID NO:9, SEQ ID NO:10, or SEQ ID NO:11;
(b) a nucleic acid sequence encoding the polypeptide of SEQ ID NO: 12, SEQ ID NO: 13, or SEQ ID NO: 14;
(c) a nucleic acid sequence having at least 85% sequence identity to the nucleic acid sequence of (a) or (b);
(d) a nucleic acid sequence complementary to a nucleic acid sequence of (a) or (b)
(e) a nucleic acid sequence that hybridizes to the sequence of (a) or (b) under conditions of 0.15 M NaCl and 70° C.

13. The method of claim 12, wherein the plant is a monocot.

14. The method of claim 12 wherein the plant is a dicot.

15. The method of claim 14, wherein the plant is Arabidopsis thaliana or Nicotiana benthamiana.

16. The method of claim 12, wherein the promoter is inducible, organelle-specific, tissue-specific, cell-specific, developmentally-specific or constitutive.

17. The method of claim 12, wherein the construct comprises at least one additional sequence chosen from the group consisting of: a regulatory sequence, a selectable marker, a screenable marker, a secretable marker, a leader sequence, and a terminator.

18. The method of claim 12, wherein the construct is inherited from a parent plant of the plant.

19. The method of claim 12, wherein the plant is an R₀ transgenic plant.

20. The method of claim 12 wherein resistance to P. syringae pv. glycinea or P. syringae pv. tomato is increased.


SEQUENCE LISTING (19 sequences)

SEQ ID NO: 1 - DNA, 1734 bp, Nicotiana benthamiana
tatagaatactcaagctatgcatccaacgcgttgggagctctcccatatgtgtcgacctgcatggcggccgcgagattcactagtgattcgcggatccagacgctgcgtttgctggctttgatgaaaaacgacttcttcaaatacaaccattttgccaacgctttctcctcttaccaaacaaacagccccccaccaaaggctgaataagaatggggagtttgagggctattctgaagaatccagaggatttatatccattggtgaagctgaagctagcggctcgacacgcggagaagcagatcccgccgtctccaaattggggcttctgttactcaatgcttcataaggtttctcgtagctttgctctcgtcattcaacaacttccagtcgagcttcgtgacgccgtgtgcattttctatttggttcttcgagcacttgacactgttgaggatgataccagcattcccaccgatgttaaagttcctattctgatctcttttcatcagcatgtttatgatcgcgaatggcatttttcatgtggtacaaaggagtacaaggttctcatggaccagttccatcatgtatcaactgcttttctggagcttaggaaacattatcagcaggcaattgaggatattaccatgaggatgggtgcaggaatggcaaaattcatatgcaaggaggtggaaacaaccgatgattatgacgaatattgtcactatgtagctgggcttgttgggctaggattgtcaaaactgttccatgcctctgagaaagaagatctggcttcagattctctctccaactccatgggtttatttcttcagaaaacaaacatcattagagattatttggaagacataaatgaagtacccaagtgccgtatgttctggccccgtgaaatatggagtaaatatgttaacaagcttgaggaattaaagtacgaggataactcggccaaagcagtgcaatgtctaaatgacatggtcactaatgctttatcacatgtagaagattggttgacttacatgtctgctttgcgtgatccttccatctttcgattctgtgctattccacaggtcatggcaattgggacattagctatgtgctacgacaacattgaagtcttcagaggagtggtaaaaatgagacgtggtctgactgctaaggtcattgaccggaccaggactattgcagatgtatatggtgctttttttgacttttcttgtatgctgaaatccaaggttaataataatgatccaaatgcaacaaaaactctgaagaggctcgaagtgatcctgaaaacttgcagagattcgggaaccttgaacaaaaggaaatcctacataatcaggagcgagcctaattacagtccagttctgattgttgtcattttcatcatactggctattattctcgcacagctatccggaaaccgatcttagacgatattttgggttacaaaaaagaagtctggtcaaggaagacagcacaagctcttggccaattgtgtgattagtgcagattttgatgtttgtaattctattgttcattaagtgatagttgcaccttttaacctgacaagataattacgaagacctatttttggtggtttgttgggtatgtacttgtttgcaagctacgtaagcaaattccaagtgttgtagagtcaccgtgatgtaataaacatgtcttttattatagtttgttcatttttgtggtaaaaaaaaaaa

SEQ ID NO: 2 - DNA, 1236 bp, Nicotiana benthamiana
atggggagtttgagggctattctgaagaatccagaggatttatatccattggtgaagctgaagctagcggctcgacacgcggagaagcagatcccgccgtctccaaattggggcttctgttactcaatgcttcataaggtttctcgtagctttgctctcgtcattcaacaacttccagtcgagcttcgtgacgccgtgtgcattttctatttggttcttcgagcacttgacactgttgaggatgataccagcattcccaccgatgttaaagttcctattctgatctcttttcatcagcatgtttatgatcgcgaatggcatttttcatgtggtacaaaggagtacaaggttctcatggaccagttccatcatgtatcaactgcttttctggagcttaggaaacattatcagcaggcaattgaggatattaccatgaggatgggtgcaggaatggcaaaattcatatgcaaggaggtggaaacaaccgatgattatgacgaatattgtcactatgtagctgggcttgttgggctaggattgtcaaaactgttccatgcctctgagaaagaagatctggcttcagattctctctccaactccatgggtttatttcttcagaaaacaaacatcattagagattatttggaagacataaatgaagtacccaagtgccgtatgttctggccccgtgaaatatggagtaaatatgttaacaagcttgaggaattaaagtacgaggataactcggccaaagcagtgcaatgtctaaatgacatggtcactaatgctttatcacatgtagaagattggttgacttacatgtctgctttgcgtgatccttccatctttcgattctgtgctattccacaggtcatggcaattgggacattagctatgtgctacgacaacattgaagtcttcagaggagtggtaaaaatgagacgtggtctgactgctaaggtcattgaccggaccaggactattgcagatgtatatggtgctttttttgacttttcttgtatgctgaaatccaaggttaataataatgatccaaatgcaacaaaaactctgaagaggctcgaagtgatcctgaaaacttgcagagattcgggaaccttgaacaaaaggaaatcctacataatcaggagcgagcctaattacagtccagttctgattgttgtcattttcatcatactggctattattctcgcacagctatccggaaaccgatcttag

SEQ ID NO: 3 - DNA, 1693 bp, Arabidopsis thaliana
aaaaaagaaagaaaaaaaattcagaagcctttgttttcatcgatgaataaattaacaaacaaaactaaaaaataggatattcgctggccgaatcgcgtcgatccacatcgcaggtgagggttcctgcaatttatccctcgtggtctctgaatctcagatcgtcgtcaacgaatcctccattttctgaatcaaaattttctggaaacaatggggagcttggggacgatgctgagatatccggatgacatatatccgctcctgaagatgaaacgagcgattgagaaagcggagaagcagatccctcctgagccacactggggtttctgctattcgatgctccacaaggtttctcgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctgcagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcagatcatggcgattggaacacttgcattatgctataacaatgaacaagtattcagaggcgttgtgaaactgaggcgaggtcttactgctaaagtcattgatcgtacaaagacaatggctgatgtctatggtgctttctatgatttttcctgcatgctgaagacaaaggttgacaagaacgatccaaatgccagtaagacactaaaccgacttgaagccgttcagaaactctgcagagacgctggagttcttcaaaacagaaaatcttatgttaatgacaaaggacaaccaaacagtgtctttattataatggttgtgattctactggccatagtctttgcatatctcagagcaaactgagtgatccatgtaagcgagtctgattgtatcaccatcattcaagatgttcagagcaaatttgagtgatgaagtaatctaggttgattcttattcacgccactgaatcctaagcaagattgtttccagaacaaacagagtttaagcatggtttagtctaaaaccatggattctattttagttactaccttcgttatctaaacgtgcatttgttcatctatttttattccttgtgtttaaagttctttctttgttt

SEQ ID NO: 4 - DNA, 1233 bp, Arabidopsis thaliana
atggggagcttggggacgatgctgagatatccggatgacatatatccgctcctgaagatgaaacgagcgattgagaaagcggagaagcagatccctcctgagccacactggggtttctgctattcgatgctccacaaggtttctcgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctgcagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcagatcatggcgattggaacacttgcattatgctataacaatgaacaagtattcagaggcgttgtgaaactgaggcgaggtcttactgctaaagtcattgatcgtacaaagacaatggctgatgtctatggtgctttctatgatttttcctgcatgctgaagacaaaggttgacaagaacgatccaaatgccagtaagacactaaaccgacttgaagccgttcagaaactctgcagagacgctggagttcttcaaaacagaaaatcttatgttaatgacaaaggacaaccaaacagtgtctttattataatggttgtgattctactggccatagtctttgcatatctcagagcaaactga

SEQ ID NO: 5 - DNA, 3627 bp, Arabidopsis thaliana
aaaaaagaaagaaaaaaaattcagaagcctttgttttcatcgatgaataaattaacaaacaaaactaaaaaataggatattcgctggccgaatcgcgtcgatccacatcgcaggtgagggttcctgcaatttatccctcgtggtctctgaatctcagatcgtcgtcaacgaatcctccattttctgaatcaaaattttctggaaacaatggggagcttggggacgatgctgagatatccggatgacatatatccgctcctgaagatgaaacgagcgattgagaaagcggagaagcagatccctcctgagccacactggggtttctgctattcgatgctccacaaggtttctcgaagcttttctctcgttattcagcaactcaacaccgagctccgtaacgccgtaagttcttctcgctctcggcctctgtgttggtcaacgtcttcaatttttggaggaatccagatatctgacactcatttattgttgacgtcggcgatgattggtttcttctgtagaatccgatggctttgagagtttgtgttccacttagttcaatatgagttatgcagattcttattaattgattctgcagtttcttctgagctgcagtaaatgcagattgtattttagtgaactatgattcgatatgtttccacatataaaatggtgtgtgtgttcgatcacatgctatcagtgtagctgtgttcagtcttgcagcattttcttattcaatttttttttttttttcattttgtaggtgtgtgtgttctacttggttctccgagctcttgatactgttggttagtgtctcttaactttgctttcaagattaccttgatacgatgaggcttctccaaaatctgctatatttctgttttgaaatgcttttatgttagagccatctttcttcttcgtgcttctgctgctaatattattttttgtacgacagaggatgatactagcataccaactgatgaaaaggttcccatcctgatagcttttcaccggcacatatacgatactgattggcattattcatgcaagtctgaagagtctcttaacatcttcatctttcacattccattaatctttgactgtctaactgacaaattttacttgttcaactttaggtggtacgaaggagtacaagattctaatggaccaatttcaccatgtttctgcagcttttttggaacttgaaaaagggttagttctaatatttgctatacgcacttctgttctactttatagattttttctgctcatgttgtctcacgtttattgagttactcaatttgttgatactcttatctgctttctgacaatactaattacctcatttttaacatatgcttttgtatatgaaggtatcaagaggctatcgaggaaattactagaagaatgggtgcagggatggccaagtttatctgccaagaggtctgtgatacagagcagttttcgtagttggatttgtttttgagttatggtgacaaaatgtcttgagctggactgaggtctctgtttcagttagaattctgtcatgcttaatcttttcataagtcataaatattggaatgcctcatgtgatgcttctatgatatgacaagtagtaattgtattgaaagtaccaaaacttatttataagttttgtaaagaaatgaaatcatttgtttaccgatccagttgatccagttttcactattatacacaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctgcaggtacaacaatgtattttgaggtgtacaatgtttcttcttcctcaaatcttttgcaatttcggacaatttctatcattgaatgatttataacactggtcttatttgcagaaaacaaacattatcagagattatcttgaggacattaatgagataccaaaatcccgcatgttttggcctcgcgagatttggggcaaatatgctgacaagcttgaggtgcatacataccactctcaagctctcatcttaggtctgatgttacccatattgactattttctgacagtgcctttttggtgatttgtaaattgtaatgtaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcaggttagagaacgaaatatcacataattgttaagtctttaaacttcacattgagaactgtttatattgttttcctcttcatatggaattaggtattctcctctttaactacgtctgcactaatatagaatgccttagaagttaatattgctttgcttgggtgtattttcttgattagatcatggcgattggaacacttgcattatgctataacaatgaacaagtattcagaggcgttgtgaaactgaggcgaggtaagatgttaagccttagaaattatgtaggccaatccctaatttctatttatgagaaaggaaaacccgttatcttttattatcaacgagcttgagaacgtgtagtagctagccttattatctgaaggctaatatcgctgaacaactatctattgtttaggtcttactgctaaagtcattgatcgtacaaagacaatggctgatgtctatggtgctttctatgatttttcctgcatgctgaagacaaaggtagtcatcagttccatgtttgtgattagtttttcactcagtttctgtttaaagaaaagaatcatatgtgaattttctatcgacatatatgacataggttgacaagaacgatccaaatgccagtaagacactaaaccgacttgaagccgttcagaaactctgcagagacgctggagttcttcaaaacaggtacagtcttttatttaatcccaaaagctgcattaaatcctctcactaagaaactggtcacaagcacctataactaatcttccaagtattctagaataggatgatattatcataatctttctttctggttgggtttctgctgaatcattgtgtctcttcctttcagaaaatcttatgttaatgacaaaggacaaccaaacagtgtctttgtaagttacatagtttcccttttccatataagtatctcatttttaaaaaaatacataaaacttatattctatatattctacccgaaaatgcagattataatggttgtgattctactggccatagtctttgcatatctcagagcaaactgagtgatccatgtaagcgagtctgattgtatcaccatcattcaagatgttcagagcaaatttgagtgatgaagtaatctaggttgattcttattcacgccactgaatcctaagcaagattgtttccagaacaaacagagtttaagcatggtttagtctaaaaccatggattctattttagttactaccttcgttatctaaacgtgcatttgttcatctatttttattccttgtgtttaaagttctttctttgttt

SEQ ID NO: 6 - DNA, 1448 bp, Arabidopsis thaliana
atgggaagcttgagtacgattttgagacatccggatgagttatatccgctcttgaagttgaaacttgcgattacgaaagctcagaagcagattccacttgagccacacttagctttctgttattcaattcttcacaaagtttctaaaagcttttctcttgttattcaacaacttggcaccgagcttcgtaacgccgtgtgtgtgttctacttgattctccgagctcttgatactgttgaggatgacacaagcgtaccagtggagatcaaagttccaattctgatagctttccaccgtcatatatacgatggtgactggcatttttcatgtggtacaaaagagtataaacttctaatggaccaatttcaccatgtttctgcagcttttctgaaacttgaaaaagggtatcaagaggctattgaagatataactaaaagaatgggtgcaggaatggccaagttcatttgcaaggaggtagaaacaattgatgactatgatgaatactgccattatgctgcaggacttgttggtttaggtttgtcaaaaatcttcattgcttcggaattagaaatactgactccagattggaagcagatttcaaattctacaggtttatttctgcagaaaacaaacattatcaaagattatcttgaagacattaatgagagaccaaagtcgcgcatgttttggcctcgtgagatttggggaaaatatgttgacaaacttgaggacttcaaaaatgaggagaaagctacaaaagcagtgcagtgtttgaatgaaatggtcactaatgcattgaatcatgttgaagattgtttgaaatccttggcttcactgcgtgatcctgcaatatttcagtcttgcgccatccctcagatcgtggcgattggaacacttgcgttatgctataacaatgtacaagtgtttagaggtgtcgtgagattgagacgaggtctaatagctaaagtcattgatcgcacaaagacaatggatgatgtctacggtgcgttctatgatttttcttgcatgctacaaacaaaggttgacaataacgatccaaatgctatgaaaacattaaaccgactcgaaaccatcaagaaattttgcaaagaaaatggaggacttcacaaaagaaaatcttatgttaacgatgaaacacaatccaaggctatctttgttgtaatgtttgtgcttctactggccatagtcgttgtatatctcaaagcaaaccaatgtaagtgactctgattttgtgaagtcatcatgaacataatttctagcttttttgaaatgtttgatcaaatcttgaattgatagtctattcaagattgtctacgacttcaaccgtcaaaatgttctgttctttctctcttccttttcagttgttctgttttctcatttttatgaagttataaattcaaccatcatgctcataatgttggttttcc

SEQ ID NO: 7 - DNA, 1137 bp, Arabidopsis thaliana
atgggaagcttgagtacgattttgagacatccggatgagttatatccgctcttgaagttgaaacttgcgattacgaaagctcagaagcagattccacttgagccacacttagctttctgttattcaattcttcacaaagtttctaaaagcttttctcttgttattcaacaacttggcaccgagcttcgtaacgccgtgtgtgtgttctacttgattctccgagctcttgatactgttgaggatgacacaagcgtaccagtggagatcaaagttccaattctgatagctttccaccgtcatatatacgatggtgactggcatttttcatgtggtacaaaagagtataaacttctaatggaccaatttcaccatgtttctgcagcttttctgaaacttgaaaaagggtatcaagaggctattgaagatataactaaaagaatgggtgcaggaatggccaagttcatttgcaaggaggtagaaacaattgatgactatgatgaatactgccattatgctgcaggacttgttggtttaggtttgtcaaaaatcttcattgcttcggaattagaaatactgactccagattggaagcagatttcaaattctacaggtttatttctgcagaaaacaaacattatcaaagattatcttgaagacattaatgagagaccaaagtcgcgcatgttttggcctcgtgagatttggggaaaatatgttgacaaacttgaggacttcaaaaatgaggagaaagctacaaaagcagtgcagtgtttgaatgaaatggtcactaatgcattgaatcatgttgaagattgtttgaaatccttggcttcactgcgtgatcctgcaatatttcagtcttgcgccatccctcagatcgtggcgattggaacacttgcgttatgctataacaatgtacaagtgtttagaggtgtcgtgagattgagacgaggtctaatagctaaagtcattgatcgcacaaagacaatggatgatgtctacggtgcgttctatgatttttcttgcatgctacaaacaaaggttgacaataacgatccaaatgctatgaaaacattaaaccgactcgaaaccatcaagaaattttgcaaagaaaatggaggacttcacaaaaggtag

SEQ ID NO: 8 - DNA, 2171 bp, Arabidopsis thaliana
atgggaagcttgagtacgattttgagacatccggatgagttatatccgctcttgaagttgaaacttgcgattacgaaagctcagaagcagattccacttgagccacacttagctttctgttattcaattcttcacaaagtttctaaaagcttttctcttgttattcaacaacttggcaccgagcttcgtaacgccgtatgtttttttttttttctatatatagctatgttacatttttagttacagcaatatttcaaacattttctttgtttttattaggtgtgtgtgttctacttgattctccgagctcttgatactgttggttagtgcttcataactatagtatccatatattacctttgatttgatgagtcttcttctccaagtcttttggttttttaattcttgatgatagaaaaagttagtatcatcttctcatatttttgctactacagaggatgacacaagcgtaccagtggagatcaaagttccaattctgatagctttccaccgtcatatatacgatggtgactggcatttttcatgtaagtaataagaagagacttttcacatcttaacctttcagatgaaattcttttcttttaacttaattactcttcattcaattttaggtggtacaaaagagtataaacttctaatggaccaatttcaccatgtttctgcagcttttctgaaacttgaaaaagggttaatttgctatatacaaattctgttctgttttttataaatgttcttgctcatttattgattgatctactgaatttggtgatactaattttatgttttgttttactaacttatatatacaaaaaaggtatcaagaggctattgaagatataactaaaagaatgggtgcaggaatggccaagttcatttgcaaggaggtttgtgatacacagaatctttgtgatgtagattttatttttttcagttttgaagttaagcatcaaaacttctgaagagctgaacttatttgtgtattggacaggtagaaacaattgatgactatgatgaatactgccattatgctgcaggacttgttggtttaggtttgtcaaaaatcttcattgcttcggaattagaaatactgactccagattggaagcagatttcaaattctacaggtttatttctgcaggtattcattacaatggtataatattgacatgtacaaggtttcttcctcaaatgttttttgcaatctttgaagtcttttgtatgttatatcttatttgctagaaaacaaacattatcaaagattatcttgaagacattaatgagagaccaaagtcgcgcatgttttggcctcgtgagatttggggaaaatatgttgacaaacttgaggtacataaacttcactctcaagctctcatctgaagatcttatattataaaaaacaaatgtgactctttctaataatgtaggacttcaaaaatgaggagaaagctacaaaagcagtgcagtgtttgaatgaaatggtcactaatgcattgaatcatgttgaagattgtttgaaatccttggcttcactgcgtgatcctgcaatatttcagtcttgcgccatccctcaggttagagaaaaaaaacaaaattcatcccctctaatctaatattttactctgcttcaaatatcaaccagagaaatgtttatgttgatgtgacattttctgtttatttggattagatcgtggcgattggaacacttgcgttatgctataacaatgtacaagtgtttagaggtgtcgtgagattgagacgaggtaaaatgttttatcttgggattggttttcaatctgacacaatttcatcatctgaatcccaaggctattaacttgtgagttatgttctatgtgaaagattatttattgacaaacaattatattgtgtaggtctaatagctaaagtcattgatcgcacaaagacaatggatgatgtctacggtgcgttctatgatttttcttgcatgctacaaacaaaggtatgtatacgttactacatttcctgtgtatagtgttggtaactagaagatgagattgtatgtgttttaatattaggttgacaataacgatccaaatgctatgaaaacattaaaccgactcgaaaccatcaagaaattttgcaaagaaaatggaggacttcacaaaaggtag

SEQ ID NO: 9 - DNA, 1470 bp, Arabidopsis thaliana
agattaaaaactctttaaaaactttttgtcataaatcaggcaaagaagtaaaagagacttaaaactctctctctctctctcttggtcttcctcactcttaacgaaaatggactctttaacactcttcttcaccggtgcactcgtcgccgtcggtatctactggttcctctgcgttctcggtccagcagagcgtaaaggcaaacgagccgtagatctctctggtggctcaatctccgccgagaaagtccaagacaactacaaacagtactggtctttcttccgccgtccaaaagaaatcgaaaccgccgagaaagttccagacttcgtcgacacattctacaatctcgtcaccgacatatacgagtggggatggggacaatccttccacttctcaccatcaatccccggaaaatctcacaaagacgccacgcgcctccacgaagagatggccgtagatctgatccaagtcaaacctggtcaaaagatcctagacgtcggatgcggtgtcggcggtccgatgcgagcgattgcatctcactcgcgagctaacgtagtcgggattacaataaacgagtatcaggtgaacagagctcgtctccacaataagaaagctggtctcgacgcgctttgcgaggtcgtgtgtggtaacttcctccagatgccgttcgatgacaacagtttcgacggtgcttattccatcgaagccacgtgtcacgcgccgaagctggaagaagtgtacgcagagatctacagggtgttgaaacccggatctatgtatgtgtcgtacgagtgggttacgacggagaaatttaaggcggaggatgacgaacacgtggaggtaatccaagggattgagagaggcgatgcgttaccagggcttagggcttacgtggatatagctgagacggctaaaaaggttgggtttgagatagtgaaggagaaggatctggcgagtccaccggctgagccgtggtggactaggcttaagatgggtaggcttgcttattggaggaatcacattgtggttcagattttgtcagcggttggagttgctcctaaaggaactgttgatgttcatgagatgttgtttaagactgctgattatttgaccagaggaggtgaaaccggaatattctctccgatgcatatgattctctgcagaaaaccggagtcaccggaggagagttcttgagaaaggtagaaaggaaacatcaccggaaaaagtatggagaattttctcaatttgtttttatttttaagttaaatcaacttggttattgtactatttttgtgttttaatttggtttgtgtttcaagaattattagtttttttttgttttgttgcatatgagaatcttactcttgatttctccgccgtagtgccggcgagacataggggattattagtatttttaagtgtgtttaagattgattaacaagttagtaaaataaaatgtacttaggtgtcga

SEQ ID NO: 10 - DNA, 1086 bp, Arabidopsis thaliana
atggactctttaacactcttcttcaccggtgcactcgtcgccgtcggtatctactggttcctctgcgttctcggtccagcagagcgtaaaggcaaacgagccgtagatctctctggtggctcaatctccgccgagaaagtccaagacaactacaaacagtactggtctttcttccgccgtccaaaagaaatcgaaaccgccgagaaagttccagacttcgtcgacacattctacaatctcgtcaccgacatatacgagtggggatggggacaatccttccacttctcaccatcaatccccggaaaatctcacaaagacgccacgcgcctccacgaagagatggccgtagatctgatccaagtcaaacctggtcaaaagatcctagacgtcggatgcggtgtcggcggtccgatgcgagcgattgcatctcactcgcgagctaacgtagtcgggattacaataaacgagtatcaggtgaacagagctcgtctccacaataagaaagctggtctcgacgcgctttgcgaggtcgtgtgtggtaacttcctccagatgccgttcgatgacaacagtttcgacggtgcttattccatcgaagccacgtgtcacgcgccgaagctggaagaagtgtacgcagagatctacagggtgttgaaacccggatctatgtatgtgtcgtacgagtgggttacgacggagaaatttaaggcggaggatgacgaacacgtggaggtaatccaagggattgagagaggcgatgcgttaccagggcttagggcttacgtggatatagctgagacggctaaaaaggttgggtttgagatagtgaaggagaaggatctggcgagtccaccggctgagccgtggtggactaggcttaagatgggtaggcttgcttattggaggaatcacattgtggttcagattttgtcagcggttggagttgctcctaaaggaactgttgatgttcatgagatgttgtttaagactgctgattatttgaccagaggaggtgaaaccggaatattctctccgatgcatatgattctctgcagaaaaccggagtcaccggaggagagttcttga

SEQ ID NO: 11 - DNA, 1470 bp, Arabidopsis thaliana; identical to SEQ ID NO: 9

SEQ ID NO: 12 - protein, 411 aa, Nicotiana benthamiana
MGSLRAILKNPEDLYPLVKLKLAARHAEKQIPPSPNWGFCYSMLHKVSRSFALVIQQLPVELRDAVCIFYLVLRALDTVEDDTSIPTDVKVPILISFHQHVYDREWHFSCGTKEYKVLMDQFHHVSTAFLELRKHYQQAIEDITMRMGAGMAKFICKEVETTDDYDEYCHYVAGLVGLGLSKLFHASEKEDLASDSLSNSMGLFLQKTNIIRDYLEDINEVPKCRMFWPREIWSKYVNKLEELKYEDNSAKAVQCLNDMVTNALSHVEDWLTYMSALRDPSIFRFCAIPQVMAIGTLAMCYDNIEVFRGVVKMRRGLTAKVIDRTRTIADVYGAFFDFSCMLKSKVNNNDPNATKTLKRLEVILKTCRDSGTLNKRKSYIIRSEPNYSPVLIVVIFIILAIILAQLSGNRS

SEQ ID NO: 13 - protein, 410 aa, Arabidopsis thaliana
MGSLGTMLRYPDDIYPLLKMKRAIEKAEKQIPPEPHWGFCYSMLHKVSRSFSLVIQQLNTELRNAVCVFYLVLRALDTVEDDTSIPTDEKVPILIAFHRHIYDTDWHYSCGTKEYKILMDQFHHVSAAFLELEKGYQEAIEEITRRMGAGMAKFICQEVETVDDYDEYCHYVAGLVGLGLSKLFLAAGSEVLTPDWEAISNSMGLFLQKTNIIRDYLEDINEIPKSRMFWPREIWGKYADKLEDLKYEENTNKSVQCLNEMVTNALMHIEDCLKYMVSLRDPSIFRFCAIPQIMAIGTLALCYNNEQVFRGVVKLRRGLTAKVIDRTKTMADVYGAFYDFSCMLKTKVDKNDPNASKTLNRLEAVQKLCRDAGVLQNRKSYVNDKGQPNSVFIIMVVILLAIVFAYLRAN

SEQ ID NO: 14 - protein, 378 aa, Arabidopsis thaliana
MGSLSTILRHPDELYPLLKLKLAITKAQKQIPLEPHLAFCYSILHKVSKSFSLVIQQLGTELRNAVCVFYLILRALDTVEDDTSVPVEIKVPILIAFHRHIYDGDWHFSCGTKEYKLLMDQFHHVSAAFLKLEKGYQEAIEDITKRMGAGMAKFICKEVETIDDYDEYCHYAAGLVGLGLSKIFIASELEILTPDWKQISNSTGLFLQKTNIIKDYLEDINERPKSRMFWPREIWGKYVDKLEDFKNEEKATKAVQCLNEMVTNALNHVEDCLKSLASLRDPAIFQSCAIPQIVAIGTLALCYNNVQVFRGVVRLRRGLIAKVIDRTKTMDDVYGAFYDFSCMLQTKVDNNDPNAMKTLNRLETIKKFCKENGGLHKR

SEQ ID NO: 15 - DNA, 798 bp, Arabidopsis thaliana
gattgagaaagcggagaagcagatccctcctgagccacactggggtttctgctattcgatgctccacaaggtttctcgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggcgatttccaattcaatgggtttatttctgcagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgc

SEQ ID NO: 16 - DNA, 52 bp, Artificial sequence
  synthetic primer
ggggacaagtttgtacaaaaaagcaggctgattgagaaagcggagaagcaga

SEQ ID NO: 17 - DNA, 53 bp, Artificial sequence
  synthetic primer
ggggaccactttgtacaagaaagctgggtgcacagaaccgaaatatggaagga

SEQ ID NO: 18 - DNA, 66 bp, Artificial sequence
  synthetic primer
ggggacaagtttgtacaaaaaagcaggcttagaaggagatagaaccatggggagcttggggacgat

SEQ ID NO: 19 - DNA, 55 bp, Artificial sequence
  synthetic primer
ggggaccactttgtacaagaaagctgggtctcagtttgctctgagatatgcaaag